(19) United States
(12) Patent Application Publication
(10) Pub. No.: US 2026/0094672 A1
(43) Pub. Date: Apr. 2, 2026

(54) SYSTEMS AND METHODS FOR TANDEM REPEAT MAPPING

(71) Applicant: PACIFIC BIOSCIENCES OF CALIFORNIA, INC., Menlo Park, CA (US)

(72) Inventors: Egor Dolzhenko, Menlo Park, CA (US); Zev N. Kronenberg, Menlo Park, CA (US); William Rowell, Menlo Park, CA (US); Michael Eberle, Menlo Park, CA (US)

(21) Appl. No.: 19/112,568

(22) PCT Filed: Sep. 22, 2023
(86) PCT No.: PCT/US2023/074918

§ 371 Date: Mar. 17, 2025

Related U.S. Application Data
(60) Provisional application No. 63/376,733, filed on Sep. 22, 2022.

(51) Int. Cl. G16B 30/10

(52) U.S. Cl. CPC G16B 30/10

(57) ABSTRACT

Systems and methods for mapping a plurality of sequence reads to a genomic region are provided. A plurality of sequence reads mappable to the genomic region are obtained. An initial Markov model for the genomic region is obtained. The initial Markov model comprises at least (i) a first repeat for a first repeat region, (ii) a second repeat for a second repeat region, and (iii) an intermediate region linking the first repeat to the second repeat. The initial Markov model is refined using the plurality of sequence reads, thereby obtaining a refined Markov model. For each respective sequence read in the plurality of sequences, the respective sequence read is used to find a highest probability path through the Markov model. This highest probability path is then used to map the respective sequence read to the genomic region.

### CROSS REFERENCE TO RELATED APPLICATIONS

This application claims priority to U.S. Provisional Patent Application Ser. No. 63/376,733, entitled “SYSTEMS AND METHODS FOR TANDEM REPEAT MAPPING,” filed Sep. 22, 2022, which is hereby incorporated by reference in its entirety for all purposes.

### BACKGROUND

Sequencing of long stretches of repeated nucleotides is notoriously difficult and yet clinically important because the length and structure of repetitive regions are diagnostic markers associated with several severe human diseases (La Spada and Taylor, 2010, “Repeat expansion disease: Progress and puzzles in disease pathogenesis,” Nature Reviews Genetics 11(4), pp. 247-258; Lopez et al., 2010 “Repeat instability as the basis for human diseases and as a potential target for therapy,” Nature Reviews Molecular Cell Biology 11(3), pp. 165-170), each of which is hereby incorporated by reference. Sequence reads of genomic regions that contain tandem repeats are particularly difficult to map back to such genomic regions because such regions are highly variable from one organism to the next. For instance, such regions are known to incur repeat expansions in which short tandem repeats within such genomic regions in some organisms become more numerous (expand) relative to other organisms in a given species. Such expansions are also known as dynamic mutations due to their instability when short tandem repeats expand beyond certain sizes. As illustrated in FIG. 4, there are over a million tandem repeats in the human genome. Moreover, tandem repeats have been linked to gene expression changes, genome instability in cancer, over 50 diseases of the nervous system including amyotrophic lateral sclerosis (ALS), fragile X syndrome (FXS), and ataxias, and autism spectrum disorders.

Tandem repeat disorders (TRDs) include a family of neuropathological disorders linked to the accumulation of short-tandem repeats (STRs; repeating DNA sequences 2-6 basepairs in length). TRDs arise with STR number expansion from normal to pathological, a number that varies by disorder. TRDs account for more than 20 heritable neuropathologies, including Huntington's disease, Kennedy's disease, myotonic dystrophy, Fragile X syndrome and several spinocerebellar ataxias. See Ellegren, 2004, “Microsatellites: simple sequences with complex evolution: Nat Rev. Genet. 5:435-445, which is hereby incorporated by reference.

Moreover, different expansion states (number of repeats) of these regions can be associated with different states of such diseases. However, identifying genomic repeat expansion states using sequence reads originating from the sequences of such genomic repeats is difficult because there are vast number of different ways in which a sequence read can be mapped onto a genomic region having tandem repeats, particularly when the genomic region has undergone some degree of genomic expansion. In fact, such genomic regions having repeats can exceed 1000 base pairs in length, leading to an exponential increase in the number of possible ways to map sequence reads to such regions. As illustrated in FIG. 5, tandem repeats in the human genome account for a disproportionate number known variants in the human genome.

Accordingly, what is needed in the art are systems and methods that are capable of accurately mapping sequence reads to genomic regions that contain tandem repeats.

### SUMMARY

The present disclosure provides, inter alia, systems, computer readable media, methods, computer implemented processes for mapping a plurality of sequence reads to genomic regions that have tandem repeats. Such systems, computer readable media, methods, computer implemented processes can be used, inter alia, to determine a status, stage, presence, or absence of any of the above-described diseases. In those subjects that are found by the disclosed systems, computer readable media, methods, computer implemented processes to have such a disease, treatment for the disease can then be provided.

Using Repeat definitions. In some embodiments, a method, for mapping a plurality of sequence reads to a genomic region is provided. In some embodiments, the method comprises obtaining, in electronic form, a plurality of sequence reads that map to the genomic region.

In some embodiments, the plurality of sequence reads have a mean length of at least 500, 600, 700, 800, 900, 1000, 1100, 1200, 1300, 1400, 1500, 1600, or 2000 residues. In some embodiments, the plurality of sequence reads comprises 1000, 2000, 5000, 10,000 sequence reads, 20,000 sequence reads, 50,000 sequence reads, 100,000 sequence reads or 1×10⁶ sequence reads.

In some embodiments, the plurality of sequence reads are generated in a single molecule sequencing-by-synthesis reaction. In some embodiments, the single molecule sequencing by synthesis reaction is a Single Molecule, Real-Time (SMRT) Sequencing reaction.

In some embodiments, a repeat definition is obtained for the genomic region. In such embodiments, the repeat region comprises at least (i) a first region comprising a first variable number of repeats of a first repeat sequence, (ii) a second region comprising a second variable number of repeats of a second repeat sequence, and (iii) a fixed interruption sequence between the first region and the second region.

In some embodiments, the repeat definition specifies that the first repeat sequence is repeated at least 1, 2, 3, 4, 5, 6, 7, 8, 9, or 10 times and that the second repeat sequence is repeated at least 1, 2, 3, 4, 5, 6, 7, 8, 9, or 10 times. In some embodiments, the repeat definition specifies that the first repeat sequence is repeated at least 10, 20, 30, 40, 50, 60, 70, 80, 90, or 100 times and that the second repeat sequence is repeated at least 10, 20, 30, 40, 50, 60, 70, 80, 90, or 100 times.

In some embodiments, the first repeat sequence has a length of between 2 and 100 residues, the fixed interruption sequence has a length of between 2 and 100 residues, and the second repeat sequence has a length of between 2 and 100 residues.

In some embodiments, for each respective sequence read in the plurality of sequences, a procedure is performed that comprises using the repeat definition to generate a corresponding graph for the respective sequence read. The corresponding graph comprises a respective plurality of nodes and a respective plurality of edge. The graph is generated by scanning the respective sequence read from a first end to a second end for perfect matches to each motif in a corresponding plurality of motifs in the repeat definition. Each node in the respective plurality of nodes represents a motif in the plurality of motifs. The plurality of motifs comprises at least a first instance of the first repeat sequence, a first instance of the second repeat sequence, an instance of the fixed interruption sequence, and a second instance of the first or second repeat sequence. Each edge in the plurality of edge connects a corresponding node of a first motif and a corresponding node of a second motif in the plurality of motifs observed to be contiguous in the respective sequence read. The corresponding graph has one or more branch points. The procedure further comprises identifying a longest path through the respective graph as the candidate segmentation for the respective sequence read. In the procedure, the longest path in the respective graph is used to map the respective sequence read to the genomic region.

In some embodiments, the mapping using the longest path comprises producing a respective plurality of segmentations in accordance with the longest path and the repeat definition, selecting a respective first segmentation in the respective plurality of segmentations having a best score as the segmentation for the respective sequence read, and using the respective first segmentation to map the respective sequence read to the genomic region. In some embodiments, the respective plurality of segmentations comprises 100, 500, 1000, 2000, 3000, 4000, 5000, 10,000, 100,000 or 1×10⁶ different segmentations.

Another aspect of the present disclosure provides a system for mapping a plurality of sequence reads to a genomic region. The system comprises a memory, input/output, and a processor coupled to the memory. The system is configured to perform a method comprising obtaining, in electronic form, the plurality of sequence reads. The method further comprises obtaining a repeat definition for the genomic region. The repeat region comprises at least (i) a first region comprising a first variable number of repeats of a first repeat sequence, (ii) a second region comprising a second variable number of repeats of a second repeat sequence, and (iii) a fixed interruption sequence between the first region and the second region. The method further comprises, for each respective sequence read in the plurality of sequences, performing a procedure that comprises using the repeat definition to generate a corresponding graph for the respective sequence read. The corresponding graph comprises a respective plurality of nodes and a respective plurality of edges. The corresponding graph is constructed by scanning the respective sequence read from a first end to a second end for perfect matches to each motif in a corresponding plurality of motifs in the repeat definition. Each node in the respective plurality of nodes represents a motif in the plurality of motifs. The plurality of motifs comprises at least a first instance of the first repeat sequence, a first instance of the second repeat sequence, an instance of the fixed interruption sequence, and a second instance of the first or second repeat sequence. Each edge in the plurality of edge connects a corresponding node of an instance of a first motif and corresponding node of an instance of a second motif in the plurality of motifs observed to be contiguous in the respective sequence read. The corresponding graph has one or more branch points. The procedure further comprises identifying a longest path through the respective graph as the candidate segmentation for the respective sequence read. The procedure further comprises using the longest path in the respective graph to map the respective sequence read to the genomic region.

Another aspect of the present disclosure provides a non-transitory computer readable storage medium. The non-transitory computer readable storage medium stores instructions, which when executed by a computer system, cause the computer system to perform a method for mapping a plurality of sequence reads to a genomic region, the method. The method comprises obtaining, in electronic form, the plurality of sequence reads. The method further comprises obtaining a repeat definition for the genomic region. The repeat region comprises at least (i) a first region comprising a first variable number of repeats of a first repeat sequence, (ii) a second region comprising a second variable number of repeats of a second repeat sequence, and (iii) a fixed interruption sequence between the first region and the second region. The method further comprises performing, for each respective sequence read in the plurality of sequences, a procedure. The procedure uses the repeat definition to generate a corresponding graph for the respective sequence read. The corresponding graph comprising a respective plurality of nodes and a respective plurality of edges. The corresponding graph is generated by scanning the respective sequence read from a first end to a second end for perfect matches to each motif in a corresponding plurality of motifs in the repeat definition. Each node in the respective plurality of nodes represents a motif in the plurality of motifs. The plurality of motifs comprises at least a first instance of the first repeat sequence, a first instance of the second repeat sequence, an instance of the fixed interruption sequence, and a second instance of the first or second repeat sequence. Each edge in the plurality of edge connects a corresponding node of an instance of a first motif and corresponding node of an instance of a second motif in the plurality of motifs observed to be contiguous in the respective sequence read. The corresponding graph has one or more branch points. The procedure further comprises identifying a longest path through the respective graph as the candidate segmentation for the respective sequence read. The procedure uses the longest path in the respective graph to map the respective sequence read to the genomic region.

Using Markov models. In some embodiments, methods for mapping a plurality of sequence reads to a genomic region are provided that make use of a computer system comprising one or more processors and a system memory. In some embodiments, the genomic region has a length of between 200 and 5000 residues, between 1000 and 8000 residues, or between 2000 and 10,000 residues. In some embodiments, the methods comprise obtaining, in electronic form, a plurality of sequence reads that map to the genomic region.

In some embodiments, the plurality of sequence reads have a mean length of at least 500, 600, 700, 800, 900, 1000, 1100, 1200, 1300, 1400, 1500, 1600, or 2000 residues. In some embodiments, the plurality of sequence reads comprises 1000, 2000, 5000, or 10,000 sequence reads.

In some embodiments, the plurality of sequence reads are generated in a single molecule sequencing-by-synthesis reaction. In some embodiments, the single molecule sequencing by synthesis reaction is a Single Molecule, Real-Time (SMRT) Sequencing reaction.

In some embodiments, the methods comprise obtaining an initial Markov model for the genomic region. The initial Markov model comprises at least (i) a first repeat for a first repeat region, (ii) a second repeat for a second repeat region, and (iii) an intermediate region linking the first repeat to the second repeat. In some embodiments, the first region comprises one or more instances of a first repeat sequence having a length of between 2 and 100 residues, the intermediate regions has a length of between 2 and 100 residues, and the second region comprises one or more instances of second repeat sequence having has a length of between 2 and 100 residues. In some embodiments, the first region further comprises one or more residues that are other than the first repeat sequence, and the second region further comprises one or more residues that are other than the second repeat sequence.

In some embodiments, the methods comprise refining the initial Markov model using the plurality of sequence reads, thereby obtaining a refined Markov model. In some embodiments, the methods comprise, for each respective sequence read in the plurality of sequences, performing a procedure. The procedure uses the respective sequence read to find a highest probability path through the Markov model. Then, the procedure uses the highest probability path to map the respective sequence read to the genomic region. In some embodiments, this mapping comprises producing a respective plurality of segmentations that are each a permutation of the highest probability path, selecting a respective first segmentation in the respective plurality of segmentations having a best score as the segmentation for the respective sequence read, and using the respective first segmentation to map the respective sequence read to the genomic region. In some embodiments, the respective plurality of segmentations comprises 100, 500, 1000, 2000, 3000, 4000, 5000, 10,000, 100,000 or 1×10⁶ different segmentations.

Another aspect of the present disclosure provides a system for mapping a plurality of sequence reads to a genomic region. The system comprises a memory, input/output, and a processor coupled to the memory. The system is configured to perform a method. The method comprises obtaining, in electronic form, the plurality of sequence reads. The method further obtains an initial Markov model for the genomic region. The initial Markov model comprises at least (i) a first repeat for a first repeat region, (ii) a second repeat for a second repeat region, and (iii) an intermediate region linking the first repeat to the second repeat. The method refines the initial Markov model using the plurality of sequence reads, thereby obtaining a refined Markov model. For each respective sequence read in the plurality of sequences, the method performs a procedure. The procedure comprises using the respective sequence read to find a highest probability path through the Markov model. The procedure uses the highest probability path to map the respective sequence read to the genomic region.

Another aspect of the present disclosure provides a non-transitory computer readable storage medium. The non-transitory computer readable storage medium stores instructions, which when executed by a computer system, cause the computer system to perform a method for mapping a plurality of sequence reads to a genomic region. The method comprises obtaining, in electronic form, the plurality of sequence reads. The method further comprises obtaining an initial Markov model for the genomic region. The initial Markov model comprises at least (i) a first repeat for a first repeat region, (ii) a second repeat for a second repeat region, and (iii) an intermediate region linking the first repeat to the second repeat. The method comprises refining the initial Markov model using the plurality of sequence reads, thereby obtaining a refined Markov model. The method further comprises, for each respective sequence read in the plurality of sequences, performing a procedure. The procedure comprises using the respective sequence read to find a highest probability path through the Markov model. The procedure further comprises using the highest probability path to map the respective sequence read to the genomic region.

### DESCRIPTION OF THE FIGURES

FIG. 1 illustrates a system for mapping a plurality of sequence reads to a genomic region having tandem repeats in accordance with some embodiments of the present disclosure.

FIGS. 2A and 2B illustrate a method for mapping a plurality of sequence reads to a genomic region using repeat definitions for the genomic region in accordance with some embodiments of the present disclosure, in which optional steps are indicated by dashed boxes.

FIGS. 3A and 3B illustrate a method for mapping a plurality of sequence reads to a genomic region using a Markov model for the genomic region in accordance with some embodiments of the present disclosure, in which optional steps are indicated by dashed boxes.

FIG. 4 shows a genomic region having a tandem repeat motif that is flanked by flanking regions.

FIG. 5 shows that while tandem repeats occur in less than 4 percent of the human genome, a disproportionate number of variants occur in genomic regions having tandem repeats.

FIG. 6 shows how functional variation in tandem repeat genomic regions can be complex, leading to alleles in such region to be highly variable in size.

FIG. 7 illustrates how the high structural complexity of many genomic tandem repeat regions, generic indel callers are insufficient for tandem repeat analysis and that accurate tandem repeat analysis requires new bioinformatics tools.

FIG. 8 summarizes bioinformatics tools for analyzing genomic tandem repeat regions including a tandem repeat genotyper tool, a tandem repeat visualizer tool, and a genom-wide tandem repeat catalog with annotations of tandem repeats with population distributions of sizes and methylation in accordance with some embodiments of the present disclosure.

FIGS. 9 and 10 illustrate the use of a repeat definition for a genotypic region that has tandem repeats, in order to assist in genotyping sequence reads that map to the genotypic regions in accordance with an embodiment of the present disclosure.

FIG. 11 illustrates sequence reads that have been mapped to the HTT gene, which includes tandem repeats, using the systems and methods of the present disclosure.

FIG. 12 illustrates the identification of an initial segmentation for an input sequence mapping to a genomic region having tandem repeats in accordance with the repeat definition for the genomic region in accordance with an embodiment of the present disclosure.

FIGS. 13A, 13B, 13C, 13D, and 13E illustrate using a repeat definition for a genomic region to generate a corresponding graph for a respective sequence read to be mapped to the genomic region, the corresponding graph comprising a respective plurality of nodes and a respective plurality of edges, by scanning the respective sequence read from a first end to a second end for perfect matches to each motif in a corresponding plurality of motifs in the repeat definition, where each node in the respective plurality of nodes represents a motif in the plurality of motifs, the plurality of motifs comprises at least a first instance of the first repeat sequence, a first instance of the second repeat sequence, an instance of the fixed interruption sequence, and a second instance of the first or second repeat sequence, each edge in the plurality of edges connects a corresponding node of an instance of a first motif and corresponding node of an instance of a second motif in the plurality of motifs observed to be contiguous in the respective sequence read, and the corresponding graph has one or more branch points, (ii) identifying a longest path through the respective graph as the candidate segmentation for the respective sequence read, and (iii) using the longest path in the respective graph to map the respective sequence read to the genomic region.

FIG. 14 illustrates using dynamic programing to find a suitable segmentation for a sequence read in accordance with an embodiment of the present disclosure.

FIG. 15 illustrates sequence reads that have been mapped to a copy of a FMR1 gene having 31 copies of a CGG repeat, using the systems and methods of the present disclosure.

FIG. 16 illustrates sequence reads that have been mapped to a copy of a CNBP gene having three adjacent repeats, using the systems and methods of the present disclosure.

FIG. 17 illustrates sequence reads that have been mapped to a copy of a RFC1 gene having three a non-reference AAGAG motif, using the systems and methods of the present disclosure.

FIG. 18 illustrates using Mendelian consistency as a measure of accuracy in accordance with an embodiment of the present disclosure.

FIG. 19 illustrates how repeat types produced using the disclosed system and method have high Mendelian consistency.

FIG. 20 illustrates how polymorphic tandem repeats at a given genomic region having repeats can have a wide range of repeat lengths.

FIG. 21 illustrates that methylation in genomic regions with tandem repeats is broadly similar to the rest of the human genome.

FIG. 22 illustrates that methylation in genomic regions with tandem repeats can exhibit a bimodal methylation pattern.

FIG. 23 illustrates how methylated mosaic FMR1 expansion between 386 and 519 CGGs, an ATXN8 expansion spanning 577 CTGs, and seven biallelic RFC1 repeat expansions with 186 to 1647 AAGGGs were discovered using the systems and methods of the present disclosure.

FIG. 24 illustrates a problematic KCNMB2 repeat locus annotated as a cluster of overlapping AT repeats.

FIG. 25 illustrates the problematic KCNMB2 repeat locus of FIG. 24 consists low-complexity motifs with identical structure ((CT)ₙSTR, AAGAGG core, and (AT)ₙSTR), where each n is an independent integer.

FIG. 26 illustrates defining the KCNMB2 repeat locus with an initial unrefined hidden Markov model comprising (i) a first repeat for a first repeat region (CT repeat), (ii) a second repeat for a second repeat region (AT repeat), and (iii) an intermediate region (VNTR core) linking the first repeat to the second repeat in accordance with an embodiment of the present disclosure.

FIG. 27 illustrates how the systems and methods of the present disclosure use the initial hidden Markov model of FIG. 26 to map sequence reads to the KCNMB2 repeat locus.

FIG. 28 illustrates how the KCNMB2 VNTR is moderately polymorphic with a mean motif length of 27-30 base papers for analyzed samples.

FIG. 29 discloses that expansions of repeats in genomic RFC1 cause cerebellar ataxia, neuropathy, vestibular areflexia syndrome.

FIG. 30 illustrates defining the RFC1 repeat locus with an initial unrefined hidden Markov model in accordance with an embodiment of the present disclosure.

FIGS. 31, 32, 33, and 34 illustrate how the systems and methods of the present disclosure use the initial hidden Markov model of FIG. 30 to map sequence reads to the RFC1 repeat locus.

FIG. 35 illustrates how the AAAAG motif is the most frequent RFC1 motif in the aligned sequence reads.

FIG. 36 illustrates how the AAAGGG motif is the second most frequent RFC1 motif in the aligned sequence reads but takes up a small proportion of most alleles.

FIG. 37 illustrates a command line interface for the alignment and visualization tools of the present disclosure.

FIGS. 38 and 39 illustrate how VCFs describe allele sequences and tandem repeats contained within them in accordance with an embodiment of the present disclosure.

FIG. 40 illustrates how genotype fields contain haplotype lengths and tandem repeat coordinates in accordance with some embodiments of the present disclosure.

FIG. 41A illustrates how the allele length (AL) field contains the length of each repeat allele in accordance with some embodiments of the present disclosure.

FIGS. 41B and 41C illustrate how the motif spans (FS) field contains the span of each tandem repeat on each allele in accordance with some embodiments of the present disclosure.

### DETAILED DESCRIPTION

The present disclosure provides, inter alia, improved processes for mapping sequence reads to genomic regions that have tandem repeats. In a first method, each sequence read is segmented in accordance with a repeat definition for the genomic region. That is, for each respective sequence read under study, a segmentation is constructed using the sequence of the respective sequence read and the repeat definition for the genomic region. In this way, each sequence read receives its own segmentation. Each such segmentation is optimized against the sequence of its corresponding sequence read leading to the mapping of the sequence reads to the genomic region. For more complex genomic regions, an initial Markov model of the genomic region is defined and then refined against the plurality of sequences. The Markov model is used to provide a segmentation for each respective sequence read in the plurality of sequence reads based on the sequence of the respective sequence read. Each such segmentation is optimized against the sequence of its corresponding sequence read leading to the mapping of the sequence reads to the genomic region.

The disclosed systems and methods allow for the accurate quantification of repeat counts at specific genomic loci. Tandem repeats (TR) are repeating sequences of two or more base pairs that are adjacent to one another and are abundant throughout the genome. Because of their repetitive nature, they are hypermutable, and they play a key role in human health and disease. See, Madsen et al., 2008, “Short tandem repeats in human exons: a target for disease mutations,” BMC genomics, 9, 410, which is hereby incorporated by reference. Expansions in repeat length in certain ranges—typically longer repeats—can become pathogenic. More than 50 diseases are known to be caused by TR expansions, and further study could reveal associations with more rare diseases that are currently unexplained. The disclosed systems and methods allow for the practical applications of accurately quantifying repeat counts as a genomic location, identifying interrupting sequences at a genomic location, determining allele phasing, and determining methylation profiles. In some embodiment multiple tandem repeat catalogs are made available to enable and simplify analysis. In some embodiments, for any given genetic region of interest (e.g., a genetic locus), the disclosed systems and methods identify the sequence reads that span the region, assigns them to haplotypes, and determines the structure of the resulting repeat alleles. In some embodiments the multiple tandem repeat catalogs include tandem repeat profiles of variable number tandem repeats that are linked to diseases such as Alzheimer's, autism, epilepsy, and ALS. See, Ryan, 2019, “Tandem repeat disorders,” Evolution, Medicine, and Public Health (1), 17; and Paulson, 2018, “Repeat expansion diseases,” Handbook of clinical neurology 147, 105-123, each of which is hereby incorporated by reference.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, or from 0% to 10%, or from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

### Definitions

As used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

As used herein, the term “allele” refers to a particular sequence of one or more nucleotides at a chromosomal locus.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All embodiments of the invention can, in the alternative, be more specifically defined by any of the transitional terms “comprising,” “consisting essentially of,” and “consisting of.”

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “locus” or “site” refers to a position within a genome, e.g., on a particular chromosome and/or having a particular orientation. In some embodiments, a locus refers to a residue, a sequence tag, or a segment's position on a reference sequence. In some embodiments, a locus refers to a single nucleotide position within a genome, e.g., on a particular chromosome. In some embodiments, a locus refers to a small group of nucleotide positions within a genome, e.g., as defined by a mutation (e.g., substitution, insertion, or deletion) of consecutive nucleotides within a cancer genome. Because normal mammalian cells have diploid genomes, a normal mammalian genome (e.g., a human genome) will generally have two copies of every locus in the genome, or at least two copies of every locus located on the autosomal chromosomes, e.g., one copy on the maternal autosomal chromosome and one copy on the paternal autosomal chromosome.

As used herein, the term “mapping” refers to assigning a read sequence to a larger sequence, e.g., a reference genome. In some embodiments, mapping is performed by alignment. For instance, the mapping of a sequence read to a reference genome determines the locus in the reference genome that best matches the sequence of the sequence read.

As used herein, the term “nucleotide” can be used to refer to a native nucleotide or analog thereof. Examples include, but are not limited to, nucleotide triphosphates (NTPs) such as ribonucleotide triphosphates (rNTPs), deoxyribonucleotide triphosphates (dNTPs), or non-natural analogs thereof such as dideoxyribonucleotide triphosphates (ddNTPs) or reversibly terminated nucleotide triphosphates (rtNTPs).

As used interchangeably herein, the terms “polynucleotide,” “nucleic acid” and “nucleic acid molecules” refer to a covalently linked sequence of nucleotides (e.g., ribonucleotides for RNA and deoxyribonucleotides for DNA) in which the 3′ position of the pentose of one nucleotide is joined by a phosphodiester group to the 5′ position of the pentose of the next. In some embodiments, nucleotides include sequences of any form of nucleic acid, including, but not limited to RNA and DNA molecules such as cell-free DNA (cfDNA) molecules. The term “polynucleotide” includes, without limitation, single- and double-stranded polynucleotides.

As used herein, the term “repeat sequence” refers to a longer nucleic acid sequence including repetitive occurrences of a shorter sequence. The shorter sequence is referred to as a “repeat unit” herein. The repetitive occurrences of the repeat unit are referred to as “counts,” “repeats,” or “copies” of the repeat unit. In many contexts, a repeat sequence is associated with a gene encoding a protein. In other situations, a repeat sequence is in a non-coding region. In some embodiments, the repeat units occur in the repeat sequence with or without breaks between the repeat units. For instance, in normal samples, the FMR1 gene tends to include an AGG break in the CGG repeats, e.g., (CGG)₅+(AGG)+(CGG)₄. The term “tandem repeat,” as used herein, refers to a repeat sequence where the repeat units are contiguous. Repeat sequences lacking breaks, as well as long repeat sequences having few breaks, are prone to repeat expansion of the associated gene, which in some cases leads to genetic diseases as the repeats expand above a particular number. In various embodiments, the repeat units include 2 to 100 nucleotides. Many repeat units widely studied are trinucleotide or hexanucleotide units. Some other repeat units that have been well studied and are applicable to the embodiments disclosed herein include but are not limited to units of 4, 5, 6, 8, 12, 33, or 42 nucleotides. See, e.g., 2001, Richards, Human Molecular Genetics, 10: 20, 2187-2194. Applications of the disclosure are not limited to the specific number of nucleotide bases described above, so long as they are relatively short compared to the repeat sequence having multiple repeats or copies of the repeat units. For example, in some instances, a repeat unit includes at least 2, 3, 6, 8, 10, 15, 20, 30, 40, or 50 nucleotides. Alternatively or additionally, in some embodiments, a repeat unit includes at most about 100, 90, 80, 70, 60, 50, 40, 30, 20, 10, 6 or 3 nucleotides. In some embodiments, a repeat sequence forms a polymorphism through evolution, development, or mutagenic conditions, creating more or less copies of the same repeat unit. This process is also referred to as “dynamic mutation” due to the unstable nature of the repeat unit number. Some repeat polymorphisms have been shown to be associated with genetic disorders and pathological symptoms. Other repeat polymorphisms are not well understood or studied. In some embodiments, the disclosed methods herein are used to identify both previously known and new, unknown repeat polymorphisms. In some embodiments, a repeat sequence polymorphism is longer than about 5 base pairs (bp), about 10 bp, about 20 bp, about 50 bp, about 100 bp, about 200 bp, about 500 bp, or about 1000 bp. In some embodiments, a repeat sequence polymorphism is longer than about 1000 bp, 2000 bp, 3000 bp, 4000 bp, 5000 bp, or more. In some embodiments, a repeat sequence polymorphism is no longer than about 10,000 bp, about 5000 bp, about 2000 bp, about 1000 bp, about 500 bp, about 100 bp, about 50 bp, about 20 bp, about 10 bp, or less.

As used herein, the terms “sequencing,” “sequence determination,” and the like refers generally to any and all biochemical processes used to determine the order of biological macromolecules such as nucleic acids or proteins. For example, in some embodiments, sequencing data includes all or a portion of the nucleotide bases in a nucleic acid molecule such as an mRNA transcript or a genomic locus.

As used herein, the term “sequence read” or “read” refers to a sequence read from a portion of a nucleic acid sample. Typically, though not necessarily, a read represents a short sequence of contiguous base pairs in the sample. In some embodiments, a read is represented symbolically by the base pair sequence (in ATCG) of the sample portion. In some cases, a read is stored in a memory device and processed as appropriate to determine whether it matches a reference sequence or meets other criteria. In some instances, a read is obtained directly from a sequencing apparatus or indirectly from stored sequence information concerning the sample. In some cases, a read is a DNA sequence of sufficient length (e.g., at least about 25 bp) that can be used to identify a larger sequence or region, e.g., that can be aligned and mapped to a chromosome or genomic region or gene. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification. In some embodiments, sequence reads are produced by any sequencing process described herein or known in the art. In some cases, reads are generated from one end of nucleic acid fragments (“single-end reads”) or from both ends of nucleic acids (e.g., paired-end reads, double-end reads). The length of the sequence read is often associated with the particular sequencing technology. High-throughput methods, for example, provide sequence reads that can vary in size from tens to hundreds of base pairs (bp).

In some embodiments the sequence reads are HiFi sequences reads. HiFi reads are produced using circular consensus sequencing (CCS) mode on PacBio long-read systems. See Wenger et al., 2019, “Accurate circular consensus long-read sequencing improves variant detection and assembly of a human genome,” Nature Biotechnology, 37, 1155-1162, which is hereby incorporated by reference.

As used herein, the term “subject” refers to a human subject as well as a non-human subject such as a mammal, an invertebrate, a vertebrate, a fungus, a yeast, a bacterium, and a virus. Although the examples herein concern humans and the language is primarily directed to human concerns, the concepts disclosed herein are applicable to genomes from any plant or animal, and are useful in the fields of veterinary medicine, animal sciences, research laboratories and such.

Unless defined otherwise, all technical and scientific terms used herein have the same meaning as is commonly understood by one of skill in the art to which this disclosure belongs. All patents and publications referred to herein are incorporated by reference in their entireties.

### EXAMPLE SYSTEM EMBODIMENTS

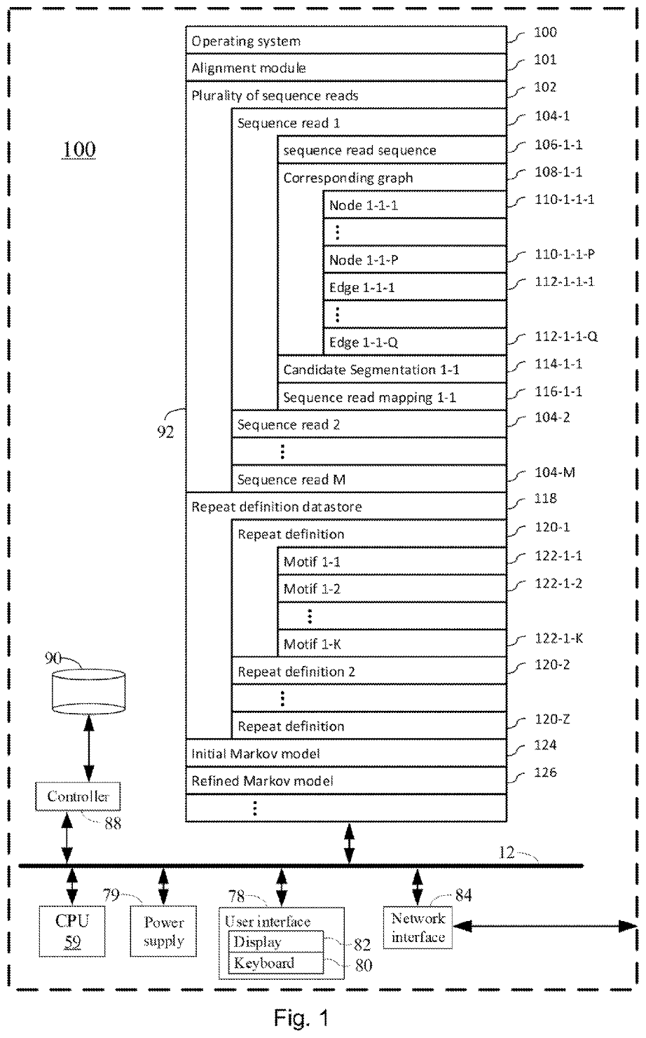

Now that an overview of some aspects of the present disclosure and some definitions used in the present disclosure have been provided, details of an exemplary system are now described in conjunction with FIG. 1 illustrates a computer system 100 for mapping a plurality of sequence reads to a genomic region.

Referring to FIG. 1, in typical embodiments, computer system 100 comprises one or more computers. For purposes of illustration in FIG. 1, the computer system 100 is represented as a single computer that includes all of the functionality of the disclosed computer system 100. However, the present disclosure is not so limited. The functionality of the computer system 100 may be spread across any number of networked computers and/or reside on each of several networked computers and/or virtual machines. One of skill in the art will appreciate that a wide array of different computer topologies are possible for the computer system 100 and all such topologies are within the scope of the present disclosure.

Turning to FIG. 1 with the foregoing in mind, the computer system 100 comprises one or more processing units (CPUs) 59, a network or other communications interface 84, a user interface 78 (e.g., including an optional display 82 and optional keyboard 80 or other form of input device), a memory 92 (e.g., random access memory, persistent memory, or combination thereof), one or more magnetic disk storage and/or persistent devices 90 optionally accessed by one or more controllers 88, one or more communication busses 12 for interconnecting the aforementioned components, and a power supply 79 for powering the aforementioned components. To the extent that components of memory 92 are not persistent, data in memory 92 can be seamlessly shared with non-volatile memory 90 or portions of memory 92 that are non-volatile/persistent using known computing techniques such as caching. Memory 92 and/or memory 90 can include mass storage that is remotely located with respect to the central processing unit(s) 59. In other words, some data stored in memory 92 and/or memory 90 may in fact be hosted on computers that are external to computer system 100 but that can be electronically accessed by the computer system 100 over an Internet, intranet, or other form of network or electronic cable using network interface 84. In some embodiments, the computer system 100 makes use of models that are run from the memory associated with one or more graphical processing units in order to improve the speed and performance of the system. In some alternative embodiments, the computer system 100 makes use of models that are run from memory 92 rather than memory associated with a graphical processing unit.

The memory 92 of the computer system 100 stores:
an optional operating system 100 that includes procedures for handling various basic system services;
an alignment module 101 for mapping a plurality of sequence reads to a genomic region;
data 102 for a plurality of sequence reads 102 including, for each sequence read 104 (e.g., 104-1, . . . , 104-M, where M is a positive integer of 3 or greater), a sequence read sequence 106, an optional corresponding graph 108 including a corresponding plurality of nodes 110 (e.g., 110-1-1-1, . . . , 110-1-1-P, where P is a positive integer) and edges 112 (e.g., 112-1-1-1, . . . , 112-1-1-Q, where Q is a positive integer), a candidate segmentation 114 (e.g., 114-1-1) and a sequence read mapping 116 (e.g., 116-1-1) (to the genomic region);
a repeat definition datastore 118 that includes, for each genomic region under consideration, a repeat definition 120 (e.g., 120-1, 120-2, . . . , 120-Z) comprising a corresponding plurality of motifs 122;
an initial Markov model 124 for segmenting sequence reads; and
a refined Markov model 126 for mapping sequence reads.

In some implementations, one or more of the above identified data elements or modules of the computer system 100 are stored in one or more of the previously mentioned memory devices, and correspond to a set of instructions for performing a function described above. The above identified data, modules or programs (e.g., sets of instructions) need not be implemented as separate software programs, procedures or modules, and thus various subsets of these modules may be combined or otherwise re-arranged in various implementations. In some implementations, the memory 92 and/or 90 optionally stores a subset of the modules and data structures identified above. Furthermore, in some embodiments the memory 92 and/or 90 stores additional modules and data structures not described above.

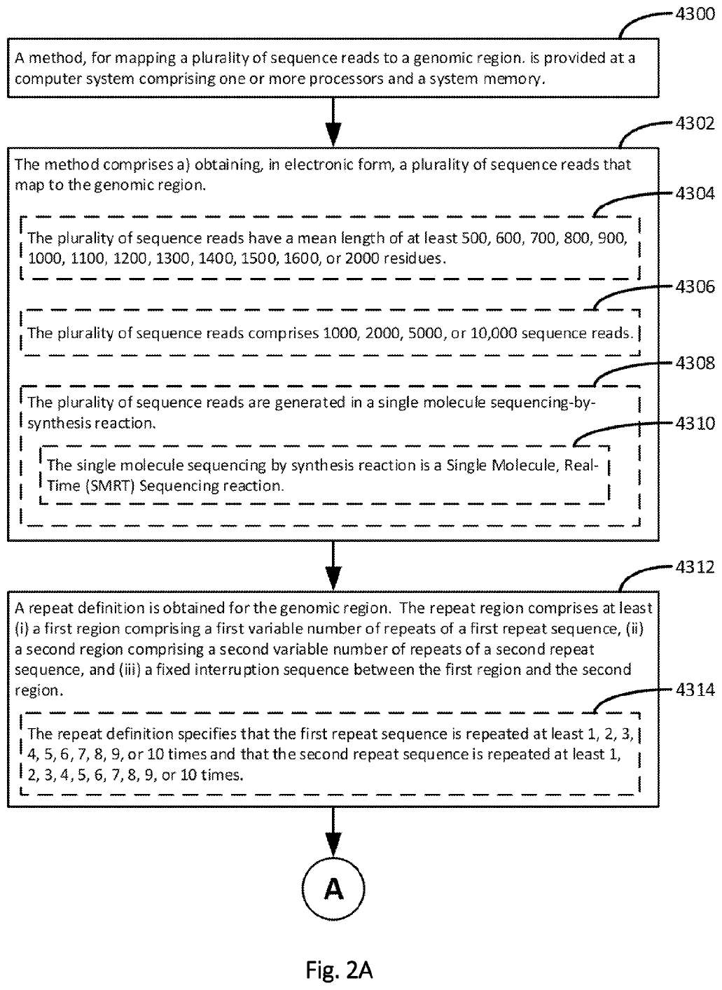

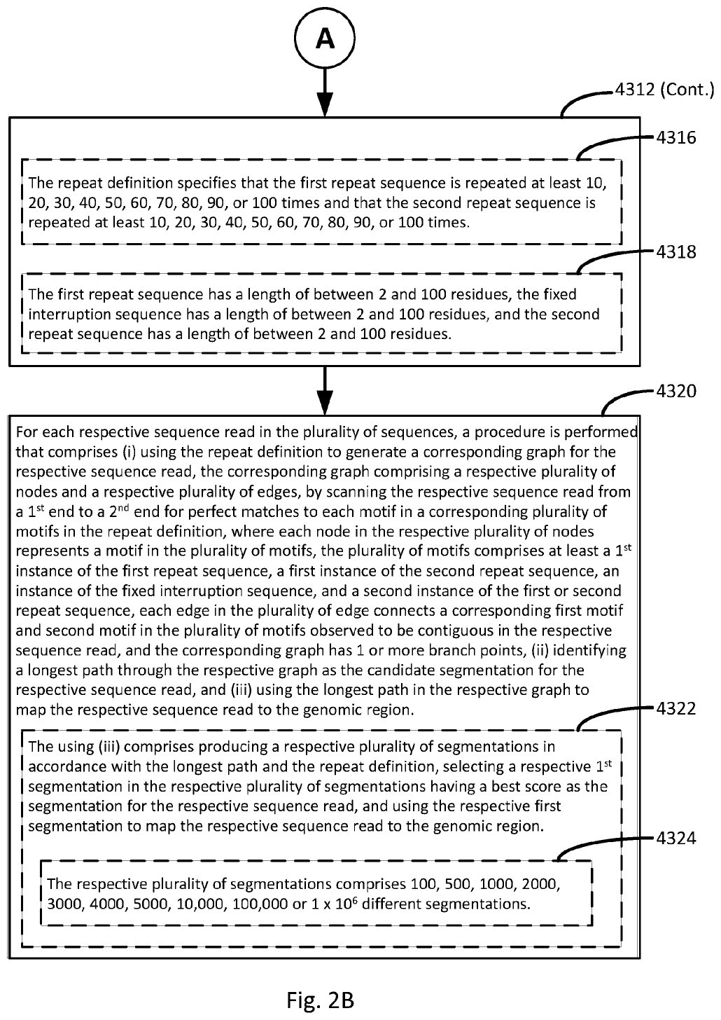

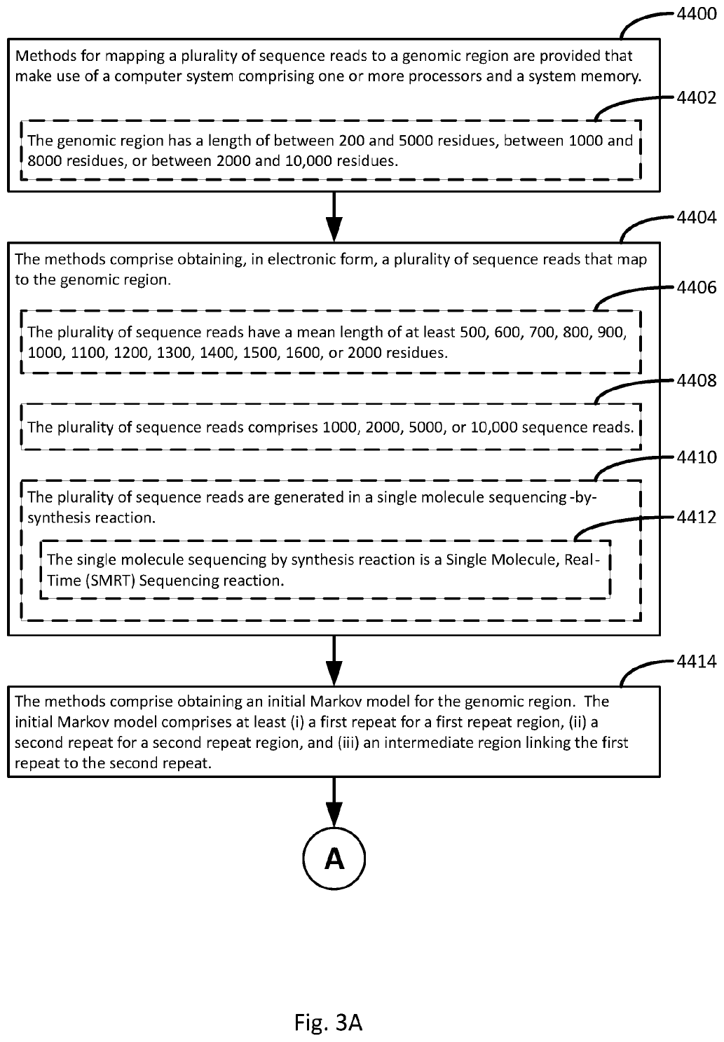

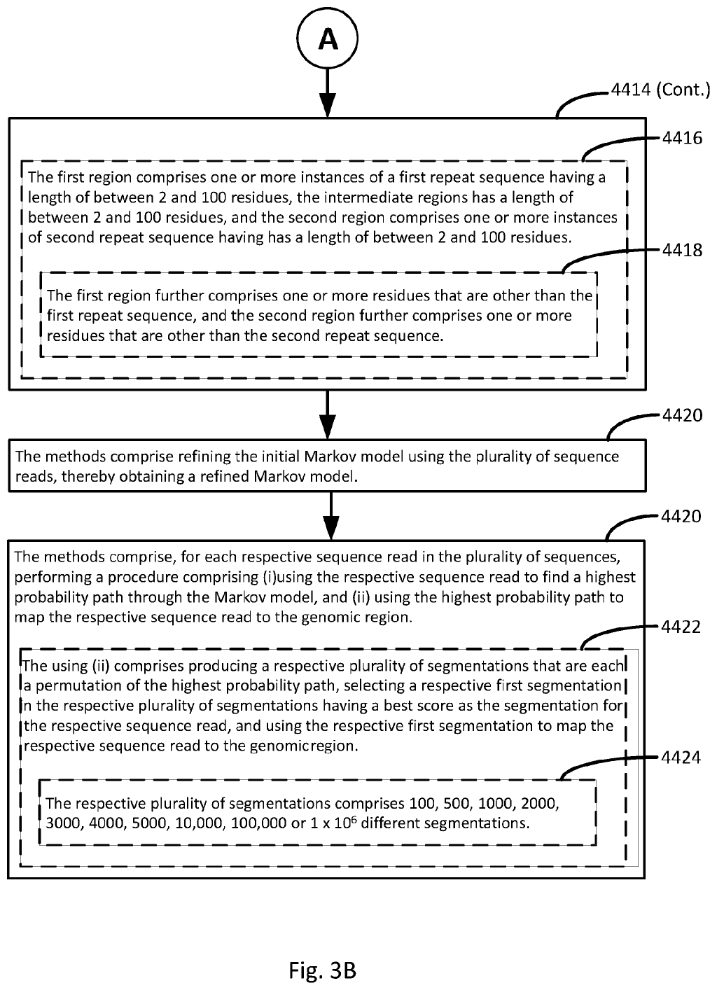

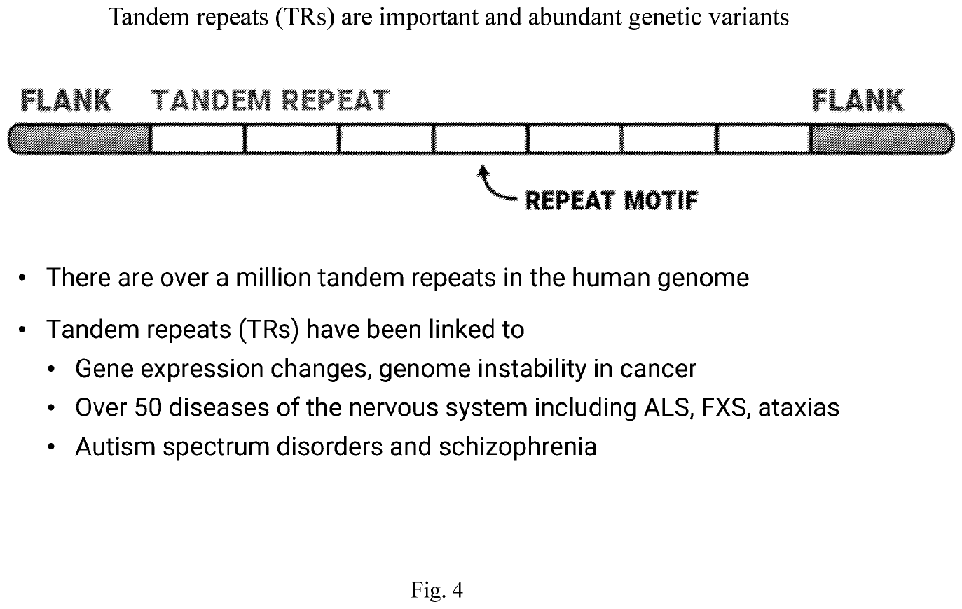

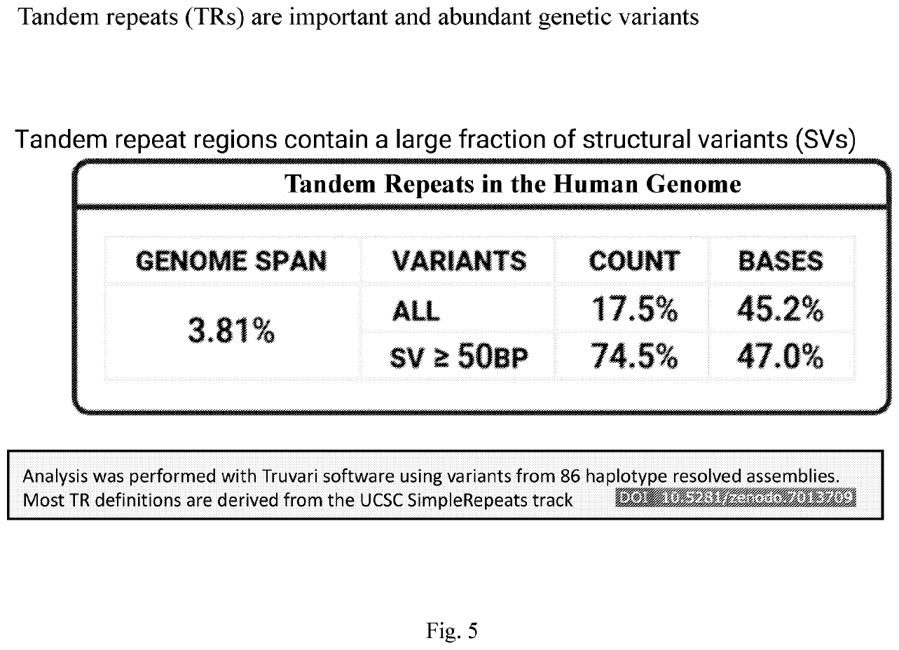

Now that a system for mapping a plurality of sequence reads to a genomic region has been disclosed, methods for performing such mapping is detailed with reference to FIGS. 2 and 3 discussed below.

### Directed Graphs.

Referring to block 4300 of FIG. 2A, in some embodiments, a method for mapping a plurality of sequence reads to a genomic region is provided at a computer system comprising one or more processors and a system memory.

Referring to block 4302, in some embodiments, the method comprises obtaining, in electronic form, a plurality of sequence reads that map to the genomic region.

Referring to block 4304, in some embodiments, the plurality of sequence reads have a mean length of at least 500, 600, 700, 800, 900, 1000, 1100, 1200, 1300, 1400, 1500, 1600, or 2000 residues. In some embodiments, the plurality of sequence reads have a mean, median or average length of about 5,000 bp to 50,000 bp long (e.g., about 5,000 bp, about 7,500 bp, about 10,000 bp, about 12,500 bp, about 15,000 bp, about 20,000 bp, about 25,000 bp, about 30,000 bp, about 35,000 bp, about 40,000 bp, about 45,000 bp, about 50,000 bp, about 55,000, about 60,000, about 65,000, about 70,000, about 75,000, or about 80,000). In some embodiments, the plurality of sequence reads have a mean, median, or average length of about 1000 bp, 2000 bp, 5000 bp, 10,000 bp, 50,000 bp or more.

Referring to block 4306, in some embodiments, the plurality of sequence reads comprises 1000, 2000, 5000, or 10,000 sequence reads. In some embodiments, the plurality of sequence reads comprises at least 1000, at least 2000, at least 3000, at least 4000, at least 5000, at least 6000, at least 7000, at least 8000, at least 9000, at least 10,000, at least 50,000, at least 100,000, at least 500,000, at least 1 million, at least 2 million, at least 3 million, at least 4 million, at least 5 million, at least 6 million, at least 7 million, at least 8 million, at least 9 million, or more sequence reads. In some embodiments, the plurality of sequence reads comprises at least 1×10⁷, at least 2×10⁷, at least 3×10⁷, at least 4×10⁷, at least 5×10⁷, at least 6×10⁷, at least 7×10⁷, at least 8×10⁷, at least 9×10⁷, at least 1×10⁸, at least 2×10⁸, at least 3×10⁸, at least 4×10⁸, at least 5×10⁸, at least 6×10⁸, at least 7×10⁸, at least 8×10⁸, at least 9×10⁸, at least 1×10⁹, or more sequence reads. In some embodiments, the plurality of sequence reads consists of no more than 5×10⁷, no more than 1×10⁷, no more than 5×10⁶, no more than 4×10⁶, no more than 3×10⁶, no more than 2×10⁶, no more than 1×10⁶, no more than 500,000, no more than 100,000, no more than 50,000, no more than 30,000, no more than 20,000, no more than 10,000, no more than 9000, no more than 8000, no more than 7000, no more than 6000, no more than 5000, no more than 4000, no more than 3000, no more than 2000, no more than 1000, or less sequence reads.

In some embodiments, the plurality of sequence reads is obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

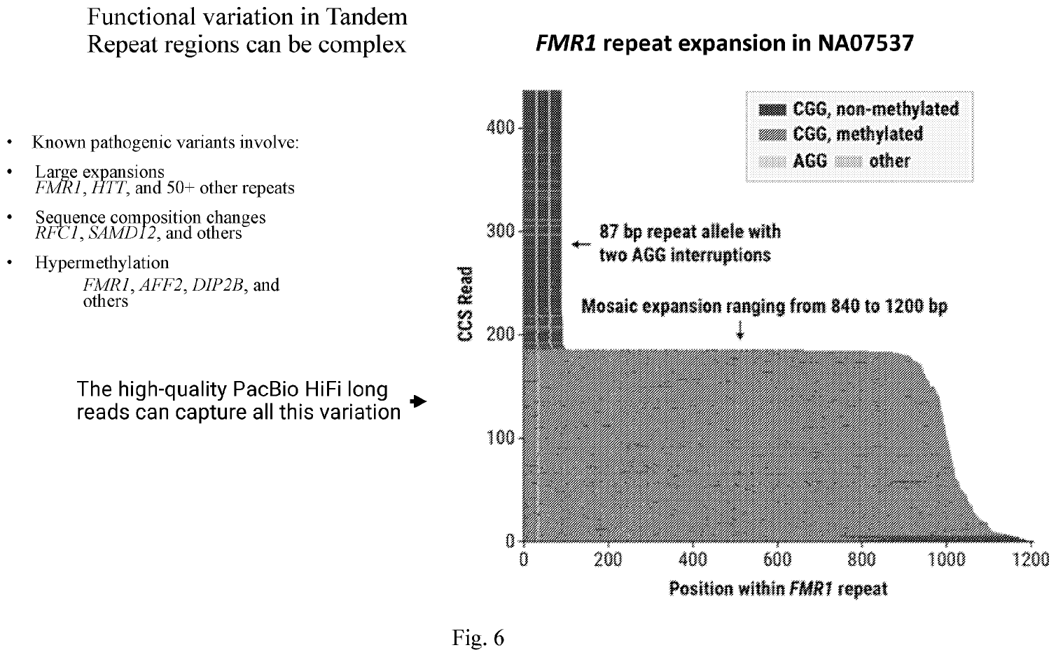

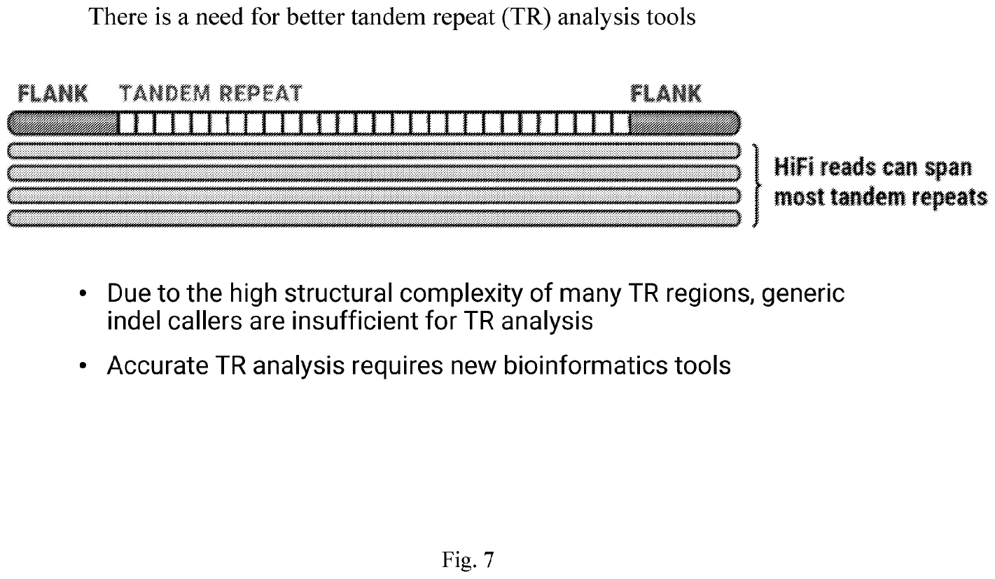

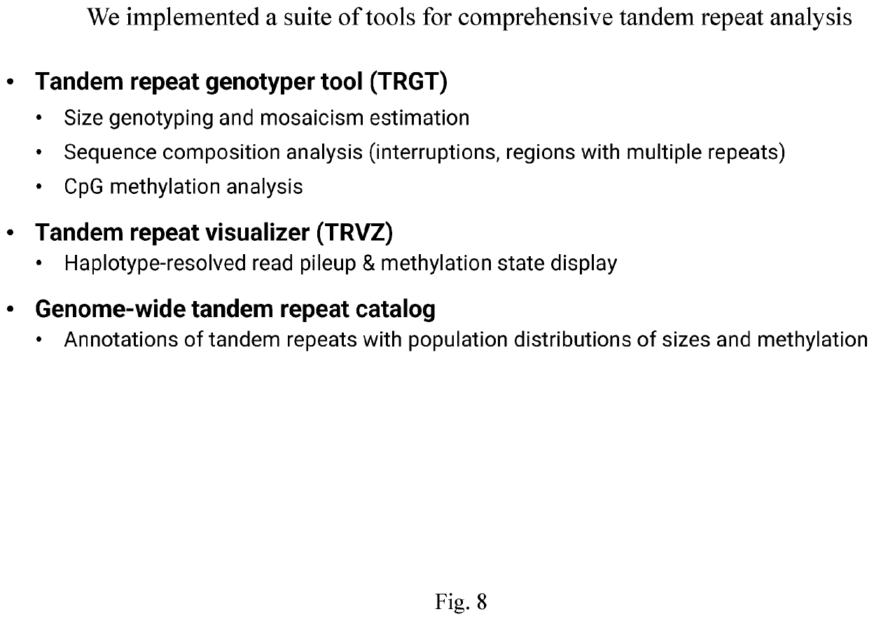

FIG. 6 illustrates how the FRMI genomic region, which has an 87 base pair allele with two AGG interruptions, can range up to 1200 base pairs in length in the examples studied for FIG. 6. Thus, for this and genomic repeat regions of similar size or larger, it is desirable to have long sequence reads, for instance sequence reads having an average length of at least 1000 base pairs, such as those disclosed in Rhoads, 2015, “PacBio Sequencing and Its Applications,” Genomics, Proteomics & Bioinformatics 13(5), pp. 278-289, which is hereby incorporated by reference, that encompass the entirety of the genomic repeat region. Referring to FIG. 7, sequence reads that encompass the entirety of the genomic repeat region are desirable because such sequence reads reduce the computational complexity of mapping to genomic repeat regions. As noted in FIG. 7, due to the high structural complexity of many genomic tandem repeat regions, conventional indel (insertion and deletion) callers are insufficient for tandem repeat analysis.

Blocks 4308-4310. Referring to block 4308, in some embodiments, the plurality of sequence reads is generated in a single molecule sequencing-by-synthesis reaction. Referring to block 4310, in some embodiments, the single molecule sequencing by synthesis reaction is a Single Molecule, Real-Time (SMRT) Sequencing reaction. In some embodiments, the plurality of sequence reads is generated in a single molecule nanopore sequencing reaction. In some embodiments, the single molecule sequencing-by-synthesis reaction is sequencing of SMRTBELL© polynucleotide substrates in Single Molecule, Real-Time (SMRT©) sequencing from Pacific Biosciences, genomic fragments used in nanopore sequencing platforms, e.g., from Oxford Nanopore Technologies, Genia, and the like, or any other convenient single molecule sequencing platform. Examples of single molecule sequencing platforms and methods that can be used to produce sequence reads used by the systems and methods of the present disclosure, in some embodiments, are found in the following U.S. patents and U.S. patent application Publications, each of which is incorporated herein by reference: U.S. Pat. No. 8,324,914, US2013/0244340, US2015/0119259, US2010/0196203, US2011/0229877, US2016/0162634, U.S. Pat. No. 7,315,019, US2009/0087850, and US2018/0023134.

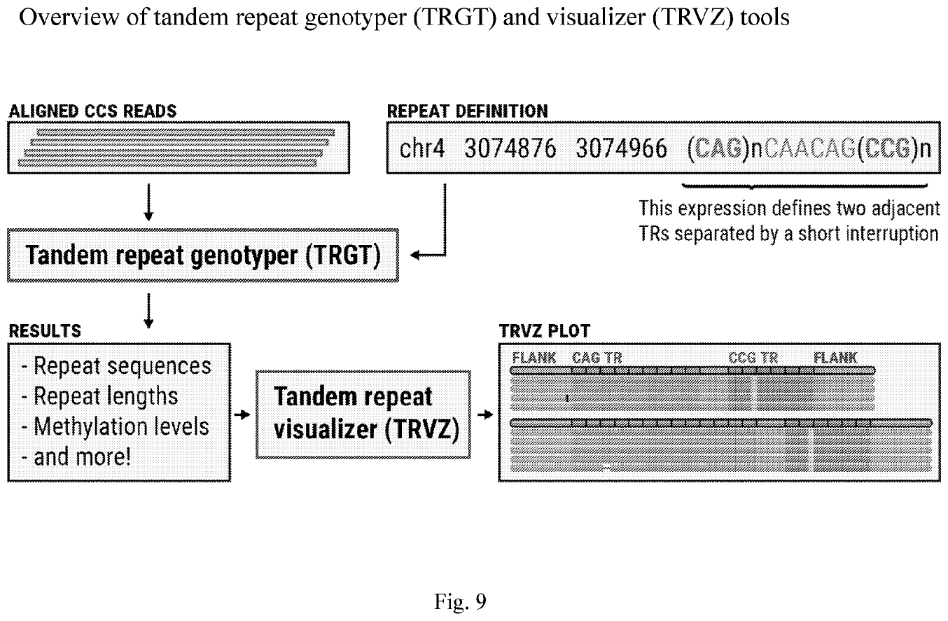

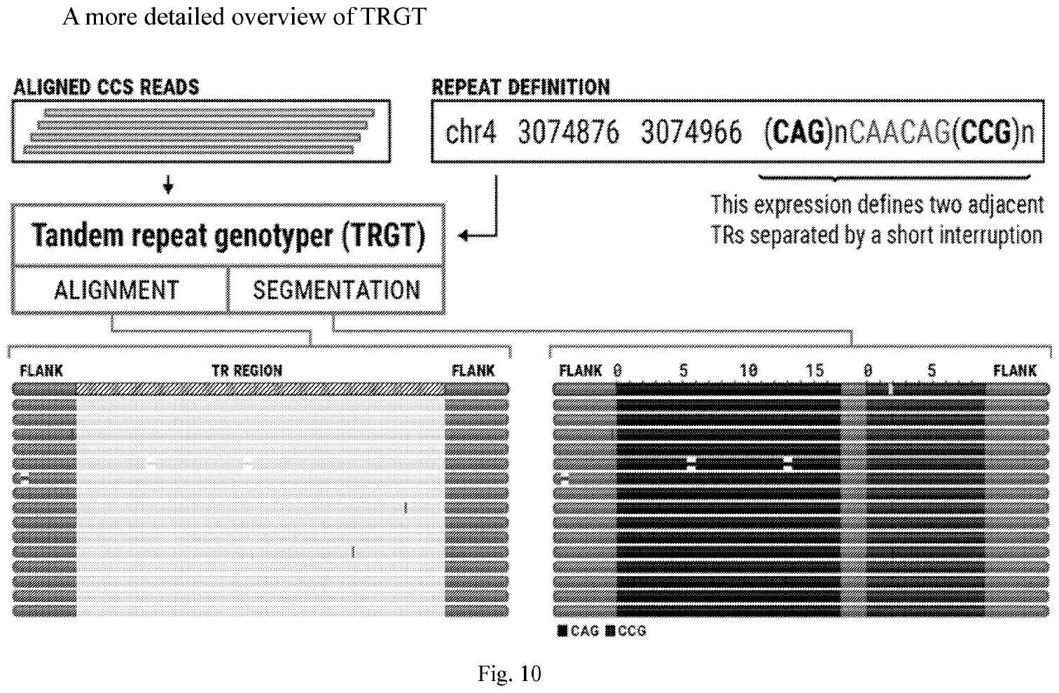

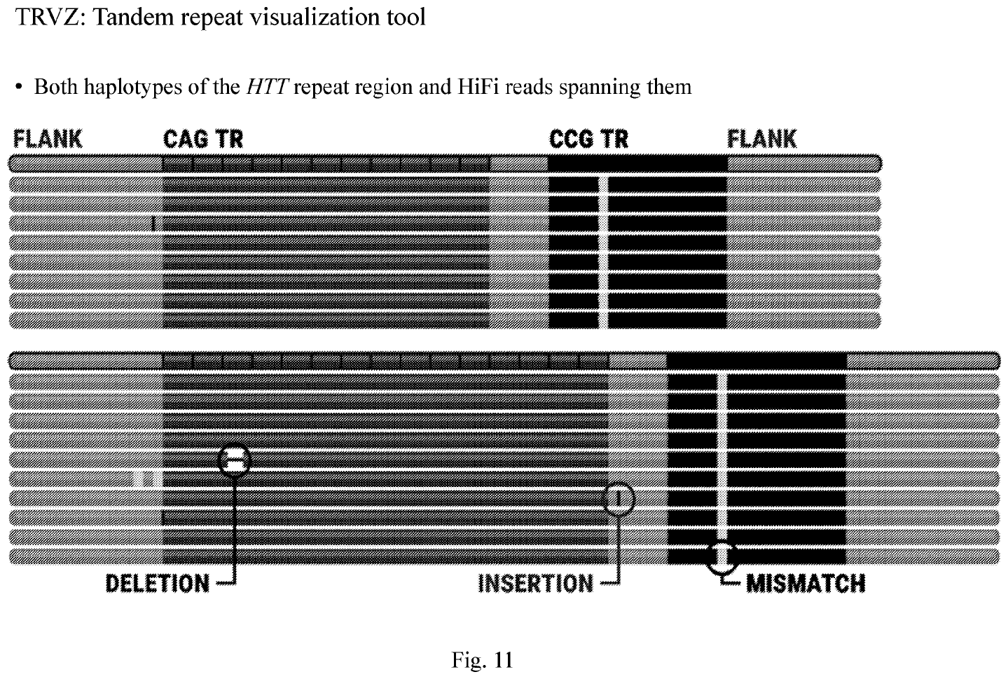

Referring to block 4312 of FIG. 2A as well as system 100 of FIG. 1, in some embodiments, a repeat definition 120 is obtained for the genomic region. In some embodiments, the repeat region comprises at least (i) a first region comprising a first variable number of repeats of a first repeat sequence, (ii) a second region comprising a second variable number of repeats of a second repeat sequence, and (iii) a fixed interruption sequence between the first region and the second region. In some embodiments a sequence read in the plurality of sequence reads has at least 2, 3, 4, 5, 6, 7, 9, 10, 11, 12, 13, 14, 15, 16, 17, 18, 19, or 20 repeats of the first repeat sequence followed by the fixed interrupt sequence followed by at least 2, 3, 4, 5, 6, 7, 9, 10, 11, 12, 13, 14, 15, 16, 17, 18, 19, or 20 repeats of the second repeat sequence. In some embodiments at least 5 sequence reads, at least 10 sequence reads, at least 15 sequence reads, at least 20 sequence reads, at least 50 sequence reads, at least 100 sequence reads, at least 250 sequence reads, at least 500 sequence reads, at least 1000 sequence reads, at least 5000 sequence reads in the plurality of sequence reads have at least 2, 3, 4, 5, 6, 7, 9, 10, 11, 12, 13, 14, 15, 16, 17, 18, 19, or 20 repeats of the first repeat sequence followed by the fixed interrupt sequence followed by at least 2, 3, 4, 5, 6, 7, 9, 10, 11, 12, 13, 14, 15, 16, 17, 18, 19, or 20 repeats of the second repeat sequence. FIG. 9 illustrates a repeat definition for a genomic region: (CAG)ₙCAACAG(CCG)ₙ. Here, each instance of “n” is the same or different positive integer. In this example, (CAG)ₙ is a motif 122 of the repeat definition 120 and is the first region comprising the first variable number of repeats of a first repeat sequence, (CCG)ₙ is another motif 122 of the repeat definition 120 and is the second region comprising the second variable number of repeats of a second repeat sequence, and CAACAG is a fixed interruption sequence between the first region and the second region. Thus a sequence in which the first instance of “n” is 2 and the second instance of “n” is three, (CAG CAG) CAACAG(CCG CCG CCG) (Seq. Id. No. 16) is encompassed by this particular repeat definition as is a sequence in which the first instance of “n” is 4 and the second instance of “n” is two, (CAG CAG CAG CAG) CAACAG(CCG CCG) (Seq. Id. No. 17). The disclosed tandem repeat genotyper of FIG. 9, also referred to herein as an embodiment of the alignment module 101 of FIG. 1, uses the repeat definition 120 to map sequence reads to the genomic region represented by the repeat definition.

While, in some embodiments, a repeat definition 120 has, at a minimum, (i) a first region comprising a first variable number of repeats of a first repeat sequence, (ii) a second region comprising a second variable number of repeats of a second repeat sequence, and (iii) a fixed interruption sequence between the first region and the second region, the present disclosure is not so limited. The repeat definition can consists of more than just two repeat regions and more than just a single fixed interruption sequence. In some embodiments, the repeat definition 120 comprises 3, 4, 5, 6, 7, 8, 9, 10, 11, 12, 13, 14, 15, 16, 17, 18, 19, 20, or more motifs 122, where each motif 122 is either a repeat or a fixed interruption sequence between two other motifs in the repeat definition. For instance, an example of a repeat definition 120 having five motifs 122 is a motif consisting of (i) a first region (motif 1) comprising a first variable number of repeats of a first repeat sequence, (ii) a second region (motif 2) comprising a second variable number of repeats of a second repeat sequence, (iii) a first fixed interruption sequence (motif 3) between the first region and the second region, (iv) a third region (motif 4) comprising a third variable number of repeats of a third repeat sequence, and (v) a second fixed interruption sequence (motif 5) between the second region and the third region. In some embodiments, the repeat definition 120 comprises between 3 and 100 motifs 122.

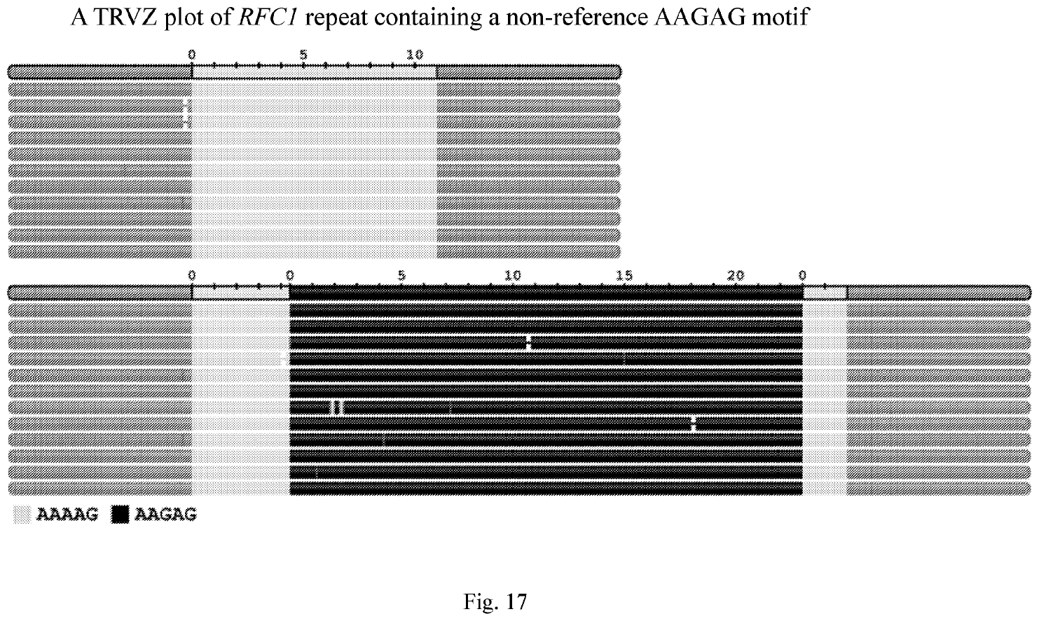

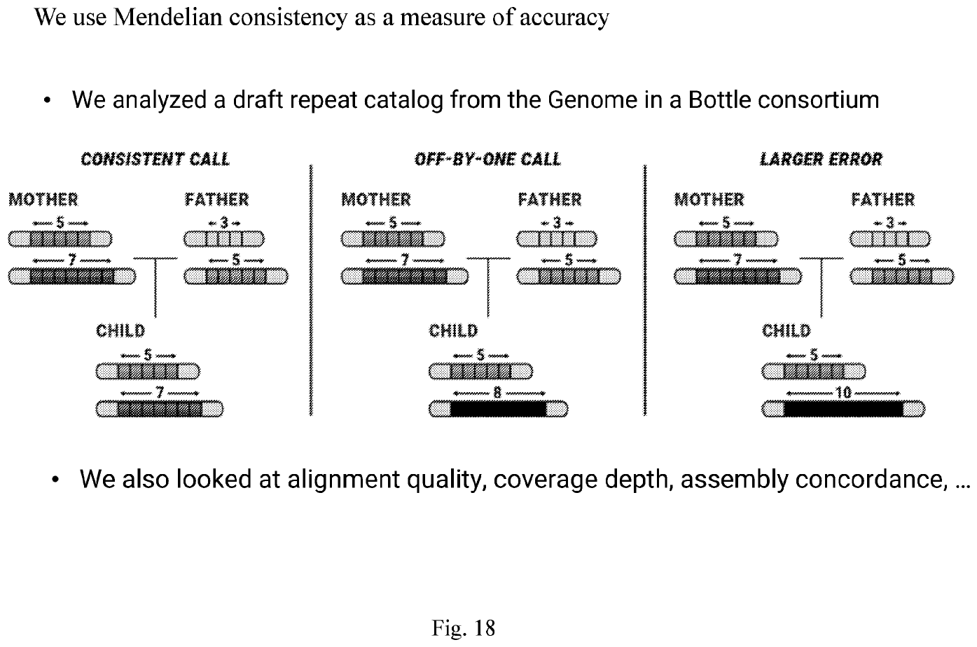

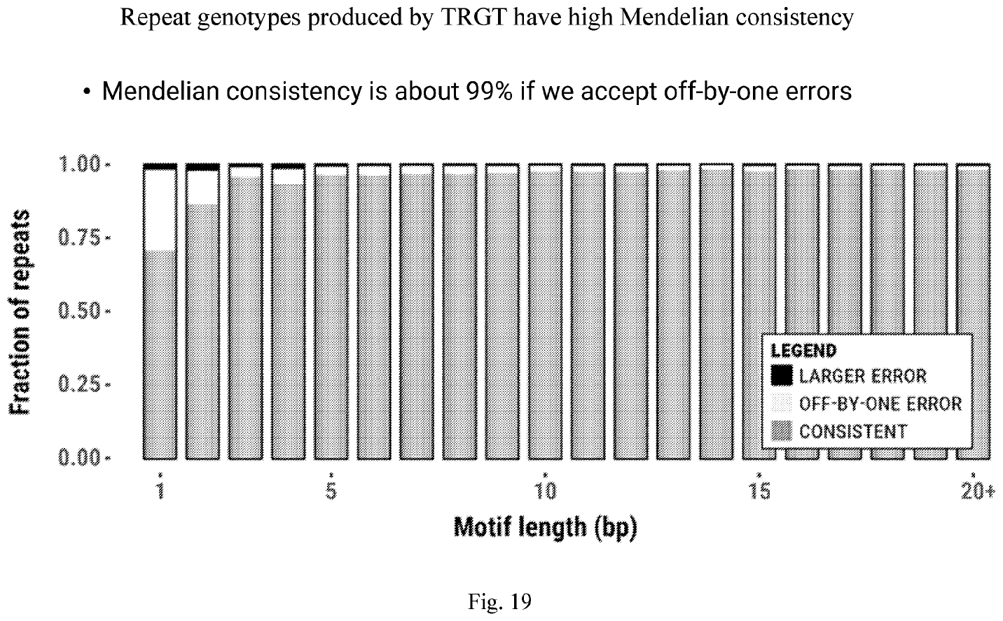

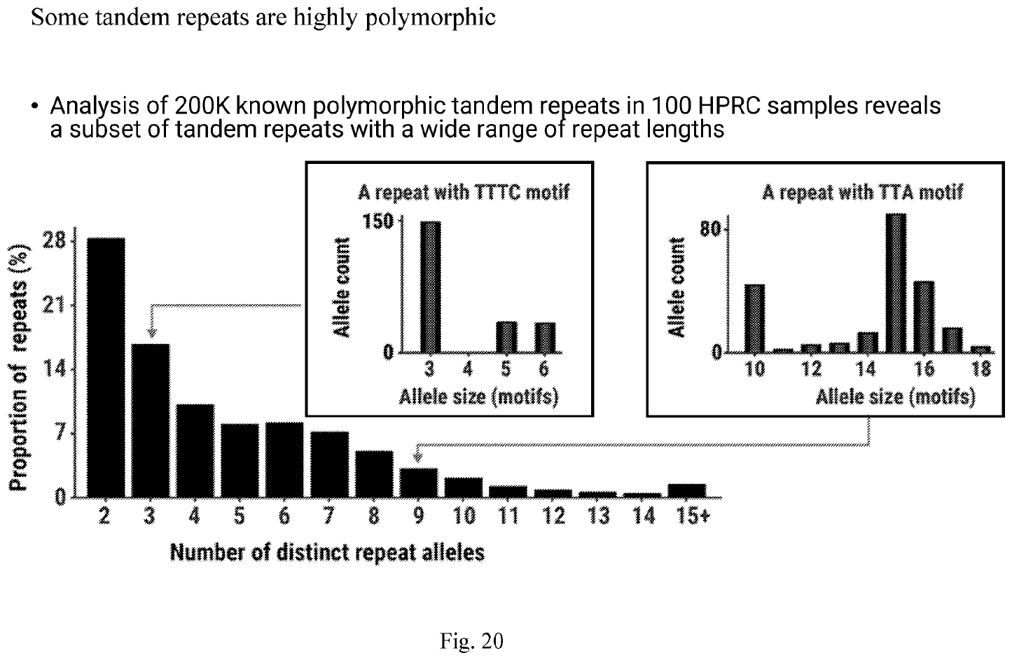

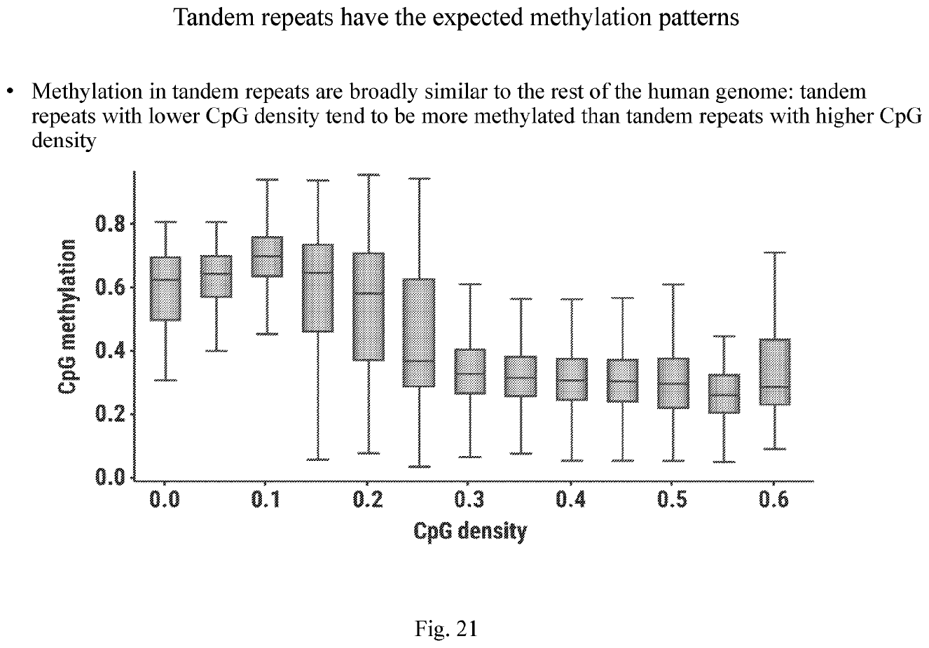

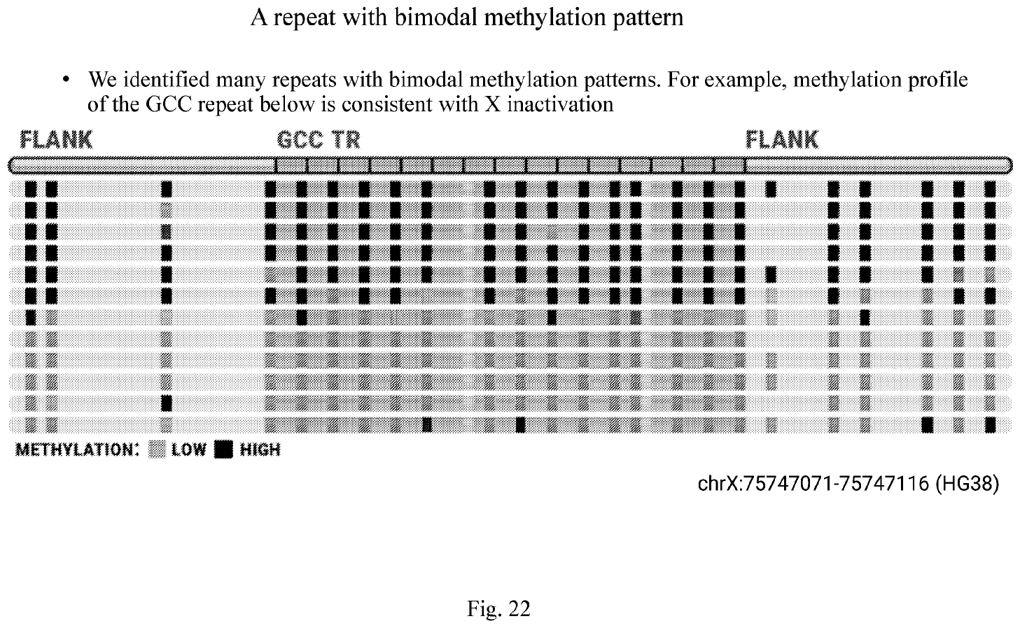

In some embodiments, a repeat region comprises three different adjacent repeat regions with no fixed interruption sequence. An example of this is illustrated for the CNBP region in FIG. 17, which includes respective adjacent CAGG, CAGA, and CA repeat regions.

In some embodiments, a repeat region comprises 3, 4, 5, 6, 7, 8, or 9 different adjacent repeat regions with no fixed interruption sequence between them. In some embodiments, a repeat region comprises three different contiguous repeat regions followed by an interruption sequence motif and followed by a fourth repeat region.

Referring to block 4314, in some embodiments, the repeat definition specifies that the first repeat sequence is repeated at least 1, 2, 3, 4, 5, 6, 7, 8, 9, or 10 times and that the second repeat sequence is repeated at least 1, 2, 3, 4, 5, 6, 7, 8, 9, or 10 times.

Referring to block 4316, in some embodiments, the repeat definition specifies that the first repeat sequence is repeated at least 10, 20, 30, 40, 50, 60, 70, 80, 90, or 100 times and that the second repeat sequence is repeated at least 10, 20, 30, 40, 50, 60, 70, 80, 90, or 100 times.

Referring to block 4318, in some embodiments, the first repeat sequence has a length of between 2 and 100 residues, the fixed interruption sequence has a length of between 2 and 100 residues, and the second repeat sequence has a length of between 2 and 100 residues.

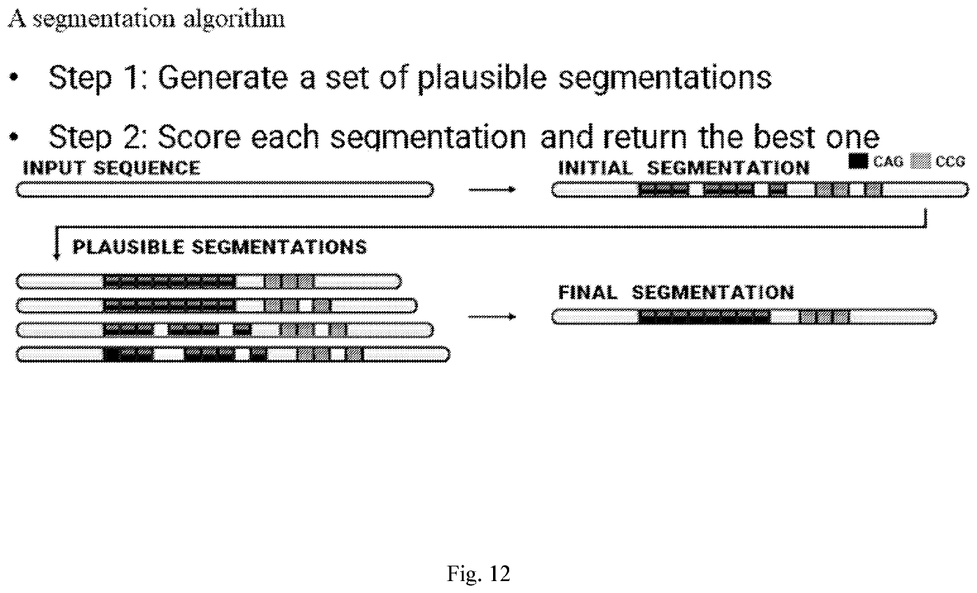

Referring to block 4320, in some embodiments, for each respective sequence read in the plurality of sequences, a procedure is performed to determine the appropriate form of the repeat definition for the genomic region to use to map the respective sequence read. A general approach to block 4320 is illustrated in FIG. 12. A set of plausible segmentations of the repeat definition 120 are generated. For example, consider the case where the repeat definition is the one illustrated in FIG. 9: (CAG)ₙCAACAG(CCG)ₙ. Here, each instance of “n” is the same or different positive integer. One plausible segmentation of (CAG)ₙCAACAG(CCG)ₙ sets the first instance of “n” to 2 and the second instance of “n” to three: (CAG CAG) CAACAG(CCG CCG CCG) (Seq. Id. No. 16). Another plausible segmentation of (CAG)ₙCAACAG(CCG)ₙ sets the first instance of “n” to 4 and the second instance of “n” is two: (CAG CAG CAG CAG) CAACAG(CCG CCG) (Seq. Id. No. 17). In accordance with FIG. 12, the input sequence of the sequence read to be mapped to a genomic region is then scored against each of the possible segmentations of the repeat definition and the repeat definition with the highest score against the sequence read is selected as the final segmentation for the sequence read. While the procedure outlined in FIG. 12 is useful for simple repeat regions, in practice there are too many possible segmentations of a repeat definition 120 to make such an approach computationally feasible.

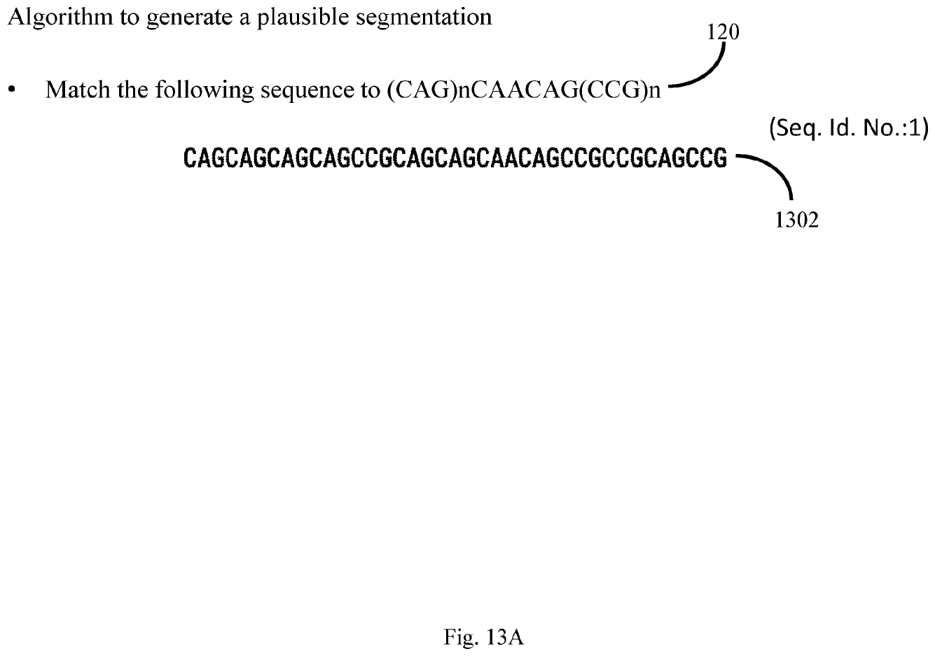

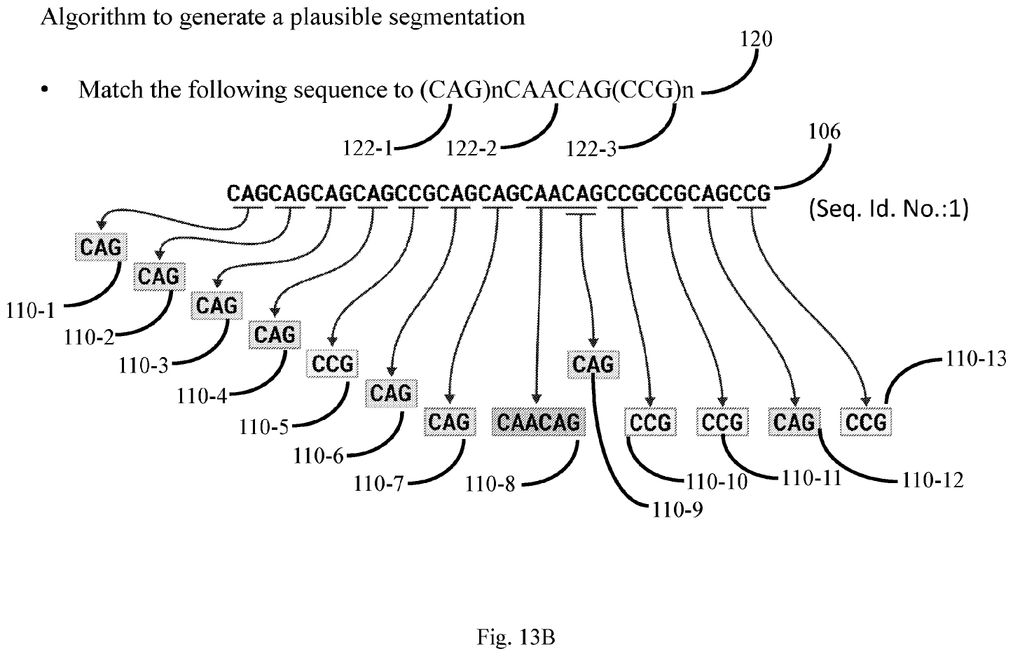

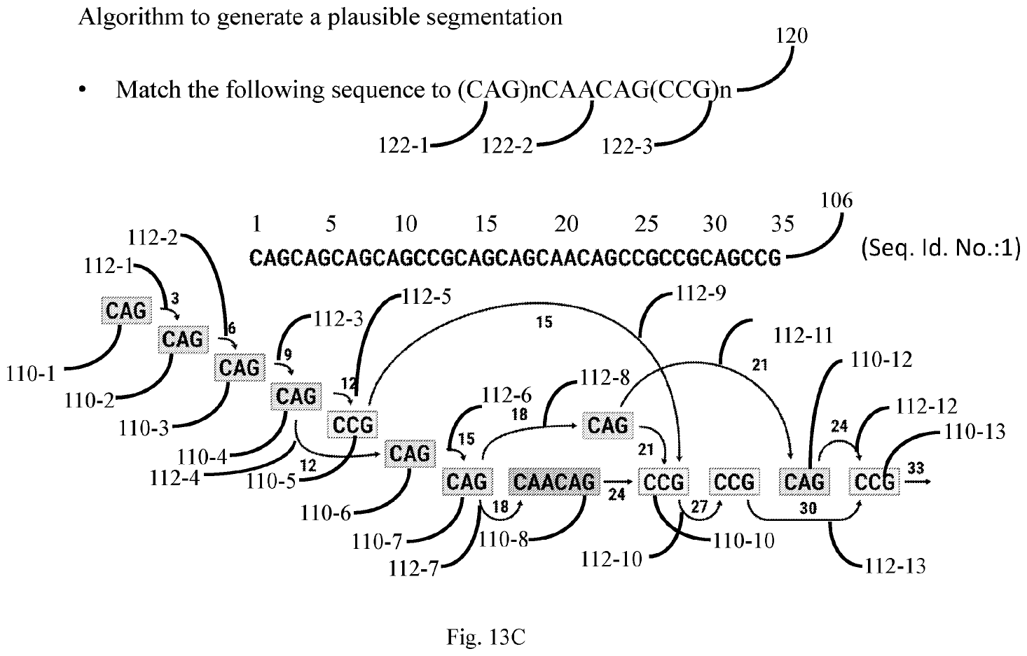

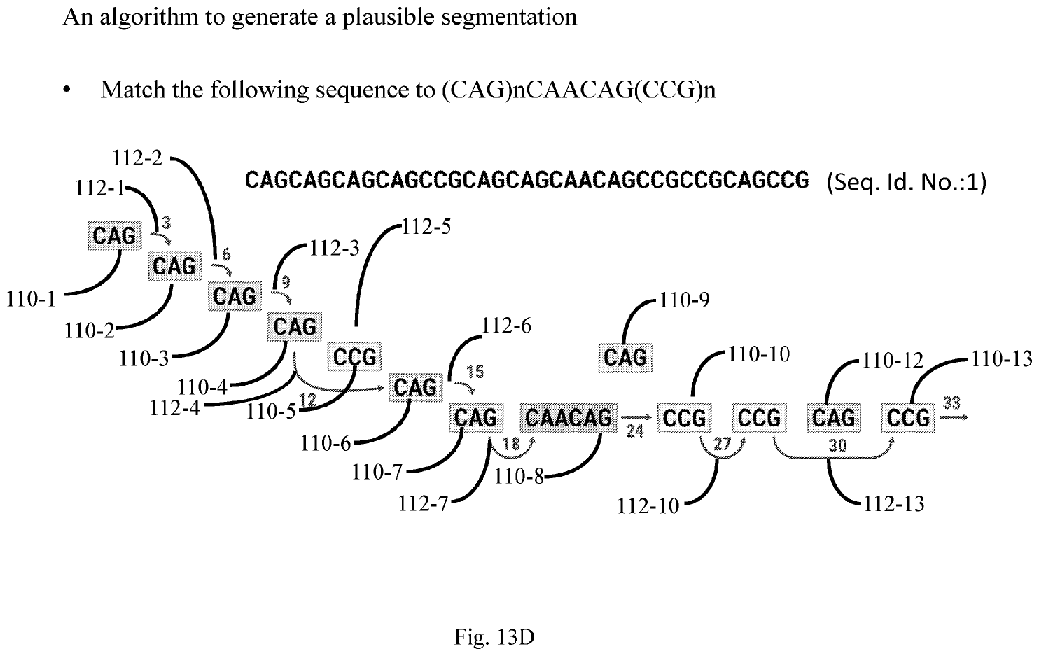

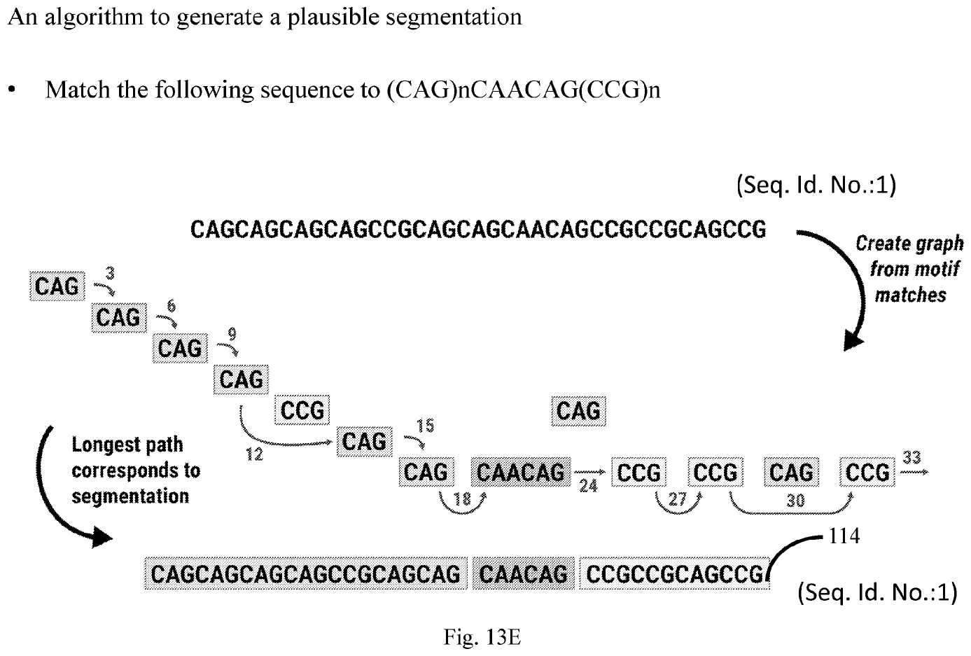

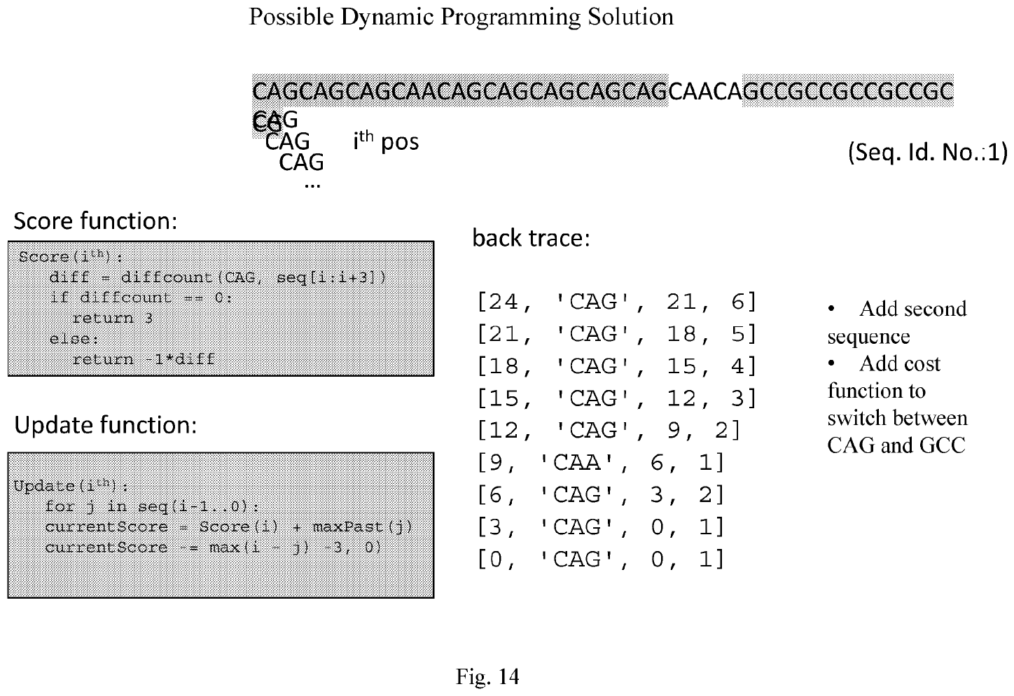

In some embodiments, the approach taken in FIG. 13 is used to reduce the segmentation search space for a repeat definition. FIG. 13A outlines the problem. The sequence read having the sequence CAGCAGCAGCAGCCGCAGCAGCAACAGCCGCCGCAGCCG (Seq. Id. No.: 1) is to be matched to the repeat definition (CAG)ₙCAACAG(CCG)ₙ in order to map the sequence read to a genomic region having repeats. The repeat definition 120 is used to generate a corresponding graph 108 for the respective sequence read 104. The corresponding graph 108 comprises a respective plurality of nodes 110 and a respective plurality of edges 112. As illustrated in FIG. 13B, to begin construction of the graph, the sequence 106 of the respective sequence read 104 is scanned from a first end to a second end for perfect matches to each motif 122 in a corresponding plurality of motifs in the repeat definition 120. The repeat definition 120 of FIG. 13B consists of three motifs: CAG (122-1), CAAACAG (122-2), and CCG (122-3). Thus, each location of each of these motifs in the sequence 106 of the respective sequence read serves as a node 110 in the corresponding graph 108. In other words, as illustrated in FIG. 13B, each node 110 in the respective plurality of nodes represents an instance of a motif 122 in the plurality of motifs. Collectively, as illustrated in FIG. 13B, the plurality of motifs comprises at least a first instance of the first repeat sequence (CAG) 122-1, a first instance of the second repeat sequence (CCG) 122-3, an instance of the fixed interruption sequence (CAACAG) 122-2, and a second instance of the first (CAG) or second (CCG) repeat sequence. Referring to FIG. 13C, each edge 112 in the plurality of edges connects a corresponding node 110 of a first motif and a corresponding node 110 of a second motif in the plurality of motifs observed to be contiguous in the respective sequence read. As illustrated in FIG. 13C, because of the highly repetitive nature of the genomic repeat region that is the source of the sequence 106 of the sequence read 104, the corresponding graph has one or more branch points. For instance, node 110-4 branches to 110-6 via edge 112-4 and to node 110-5 via edge 112-5.

In some embodiments the graph 108 is directional (e.g., from 5′ to 3′ end of the sequence 106 of the corresponding sequence read 104, or from the 3′ to 5′ end of the sequence 106 of the corresponding sequence read 104). Moreover, each node 110 in the plurality of nodes is connected to at least one other node in the plurality of nodes by an edge 112.

In some embodiments the graph 108 is a directed graph. In some embodiments, the directed graph is an acyclic graph (DAG) that has a direction as well as a lack of cycles. That is, the graph consists of finitely many nodes and edges, with each edge directed from one node to another, such that there is no way to start at any node v and follow a consistently-directed sequence of edges that eventually loops back to v again. Equivalently, a DAG is a directed graph that has a topological ordering, a sequence of the vertices such that every edge is directed from earlier to later in the sequence 106 of the corresponding sequence read 104.

In FIG. 13C, it is seen that edge 112-1 is annotated with the value “3” while edge 119-9 is annotated with the value “15”. Each of these annotations, and the annotations for the other edges in FIG. 13C, indicates the relative start point of the destination node in sequence 106 relative to the start point of the origination node in sequence 106 in nucleotide. For instance, in the case of edge 112-1, the origination node is node 110-1 and the destination node is 110-2. The “3” label on edge 112-1 between these two nodes indicates that the beginning of the motif 122 of the destination node 110-2 is displaced by three residues from the beginning of the motif 122 of the origination node 110-1 in the sequence 106 of the respective sequence read 104. In the case of FIG. 13C, the directed graph is in the direction of 5′ to 3′ of sequence 106, and thus the “3” label on edge 112-1 between these two nodes indicates that the beginning of the motif 122 of the destination node 110-2 is three residues downstream from the beginning of the motif 122 of the origination node 110-1 in sequence 106. Thus, according to edge 112-1, if motif 110-1 begins at position 1 of sequence 106, motif 110-2 begins at position 4 of sequence 106.

In some embodiments, there is 2, 3, 4, 5, 6, 7, 8, 9, 10, 11, 12, 13, 14, 15, 16, 17, 18, 19, 20, 50, 100, 1000, 10,000 or 1×10⁶ or more paths through the respective graph for a corresponding sequence read in the plurality of sequence reads that can be used as the segmentation of repeat definition 120 for the respective sequence read 104. In some embodiments, there are 2, 3, 4, 5, 6, 7, 8, 9, 10, 11, 12, 13, 14, 15, 16, 17, 18, 19, 20, 50, 100, 1000, 10,000, 1×10⁶ or more paths through each respective graph for each corresponding sequence read in the plurality of sequence reads.

In some embodiments, the corresponding graph for a respective sequence read in the plurality of sequence reads comprises 2, 3, 4, 5, 6, 7, 9, 10, 11, 12, 13, 14, 15, 16, 17, 18, 19, 20 or more nodes and 2, 3, 4, 5, 6, 7, 9, 10, 11, 12, 13, 14, 15, 16, 17, 18, 19, 20 or more edges. In some embodiments, the corresponding graph of each respective sequence read in the plurality of sequence reads comprises 2, 3, 4, 5, 6, 7, 9, 10, 11, 12, 13, 14, 15, 16, 17, 18, 19, 20 or more nodes and 2, 3, 4, 5, 6, 7, 9, 10, 11, 12, 13, 14, 15, 16, 17, 18, 19, 20 or more edges.

With the graph 108 for the sequence 106 of the sequence read 104 using motifs 122 found in the repeat definition 120 for the genomic region that the sequence read is to be mapped to constructed as illustrated in FIG. 13C, attention turns to determining which path through the graph should be used as the segmentation of repeat definition 120 for the respective sequence read 104. As illustrated in FIG. 13C, there are multiple branch points in the graph and thus there are multiple paths in the graph that each represent traversal between position 1 and position 34 of the sequence 106 of the respective sequence read 104. Each such path represents a potential segmentation of the repeat definition 120 in accordance with the sequence 106 of the respective sequence read 106. For instance, one set of paths flow though edge 112-8 while another set of paths flow through edge 112-7 since node 110-7 represent a branch point in the graph. FIGS. 13D and 13E illustrates one such path through the graph. It is noted that this path does not pass through nodes 110-9 or 110-12. The path illustrated in FIG. 13D represents the longest path through the respective graph of FIG. 13C and thus, in accordance with block 4320 of FIG. 2B, is identified as the candidate segmentation 114 for the respective sequence read 104. This longest path in the respective graph is then used to map the respective sequence read to the genomic region. In some embodiments the graph includes 10 or more paths, 100 or more paths, 1000 or more paths, 10,000 or more paths, 100,000 or more paths or 1×10⁶ or more paths, each of which is a possible segmentation for the respective sequence read. Thus, in such embodiments, the length of each of these paths is evaluated to determine which path is the longest path.

Referring to block 4322, in some embodiments, the use of the candidate segmentation 114, such as the candidate segmentation illustrated in FIG. 13E, comprises producing a respective plurality of segmentations in accordance with the longest path and the repeat definition, selecting a respective first segmentation in the respective plurality of segmentations having a best score as the segmentation for the respective sequence read, and using the respective first segmentation to map the respective sequence read to the genomic region. For instance, a plurality of segmentations based on the segmentation illustrated in FIG. 13E can be generated by adding a limited number of instances of motifs 122 specified by the repeat definition 120 and in accordance with the repeat definition. Thus, referring to block 4324, in some embodiments, the respective plurality of segmentations to be considered based on the longest path in the corresponding graph 108 comprises 100, 500, 1000, 2000, 3000, 4000, 5000, 10,000, 100,000, 1×10⁶, or more different segmentations.

The above example illustrates how the mapping of sequence reads onto genomic repeat regions cannot be mentally performed. The approach generally outlined in FIG. 12, without a graph, would take days of computation on high speed computers for repeat definitions that comprise at least (i) a first region comprising a first variable number of repeats of a first repeat sequence, (ii) a second region comprising a second variable number of repeats of a second repeat sequence, and (iii) a fixed interruption sequence between the first region and the second region (e.g., where the repeat definition specifies that the first repeat sequence is repeated at least 1, 2, 3, 4, 5, 6, 7, 8, 9, or 10 times and that the second repeat sequence is repeated at least 1, 2, 3, 4, 5, 6, 7, 8, 9, or 10 times). Such computations would be to determine the best segmentation, given the repeat definition 120 for the sequence 106 of a given sequence read 104. While the longest path through a corresponding graph 108, as illustrated in FIG. 13 reduces, by orders of magnitude, the astronomical number of possible segmentations that the brute force approach considers, it is still the case that optimization of the segmentation given by the longest path is needed resulting in the need to evaluate 100, 500, 1000, 2000, 3000, 4000, 5000, 10,000, 100,000, 1×10⁶, or more different segmentations for each sequence read based on the longest path for each such sequence read through its corresponding graph. Each such computation requires a scoring of the sequence 106 of the sequence read 104 to the sequence of the candidate segmentation to find the best score. Each such comparison requires matching the sequence 104 of the sequence read to the sequence of the candidate sequence. In some embodiments, the segmentation of the longest path with deletions, insertions and gaps introduced are also considered in order to map the sequence read to the genomic region, adding still more complexity to the mapping. This is further in the context that typical practical applications require 10, 100, 500 1000, 2000, 5000, 10,000, or more sequence reads mapping to the genomic region. In some embodiments, a graph 108 is constructed for each such sequence read in accordance with block 4320, further adding the complexity of the task involved, and the inability for it to be mentally performed.

In some embodiments, it can be difficult to resolve variation in tandem repeat (TR) regions based on the repeat sequence alone. One example is measuring methylation of homozygous repeats: if a repeat is homozygous, the reads and their methylation levels can't be assigned to alleles based on the repeat sequence alone. Another example is genotyping repeats with mosaic alleles. Such alleles give rise to reads supporting a range of repeat lengths making it difficult to determine their allele of origin. In such embodiments, using single nucleotide polymorphisms (SNPs) surrounding the repeat are used by the alignment module 101 to overcome these issues. These flanking SNPs provide independent evidence that allows for the assignment of sequence reads to alleles and subsequently genotype repeats and determination of their allele-specific methylation.

In some embodiments, for modeling purposes, each sequence read 104 r spanning the repeat is associated with a vector of ones and zeros indicating presence or absence of each single nucleotide polymorphism that the sequence read overlaps. That is, r[k]=1 if the sequence read r contains k^{th} SNP and r[k]=0 otherwise. A local haplotype is similarly defined as a vector of zeros and ones. The genotype consists of a pair of local haplotypes G=(H₁, H₂). The posterior probability of the genotype G is evaluated given the set of observed sequence reads in accordance with the following model for genotyping SNPs:

$$P ( G \vert R ) \sim P ( R \vert G ) \cdot P ( G ) ,$$

where P(R|G) is the likelihood of observing reads R given the genotype G and P(G) is the prior probability of the genotype G. Furthermore,

$$P ( R \vert G ) = {\prod }_{r ∈ R} P ( r \vert G = ( {H}_{1} , {H}_{2} ) ) = {\prod }_{r ∈ R} \frac{P ( r \vert {H}_{1} ) + P ( r \vert {H}_{2} )}{2} .$$

Here P(r|Hᵢ)=ΠIP(k|r, Hᵢ) where P(k|r, Hᵢ)=p if r[k]=Hᵢ[k] and P(k|r, Hᵢ)=1−p otherwise. The genotype probabilities P(G) can be estimated by genotyping repeats in control cohorts. This model for genotyping is described in Li et al., 2009, “SNP detection for massively parallel whole-genome resequencing,” Genome Research 19:1124-132, which is hereby incorporated by reference. Using this model, in some embodiments, the alignment module 101 determines the most likely genotype G=(H₁, H₂) and the corresponding assignment of each sequence read r to either H₁ or H₂. Finally, in such embodiments, the consensus sequence for each repeat allele is calculated from the reads assigned to the corresponding local haplotype. In some embodiments the methods of FIGS. 2A and 2B map sequence reads that have a non-reference motif to a genomic region that includes the non-reference motif. This arises in situations where the source subject of the sequence reads has an insertion at that genomic region that is not documented in references for the genomic region or is otherwise uncommon such that the motif is not included in the repeat definition 120 for the genomic region. For instance, FIG. 17 illustrates an example where sequence reads that included a non-reference AAGAG motif were successfully mapped to a RFC1 genomic region in accordance with the methods of FIGS. 2A and 2B even though the repeat definition 120 used did not include the motif AAGAG. In some embodiments, the plurality of sequence reads comprises 1, 2, 3, 4, 5, 6, 7, 8, 9, or 10 or more motifs not present in the repeat definition, where each such motif is between 1 residue and 20 residues in length and is repeated between 1 and 100 times at least some of the sequence reads in the plurality of sequence reads. In some embodiments, between 5 and 40 percent of the sequence of at least 10 percent of the sequence reads in the plurality of sequence reads arise from motifs that are not present in the repeat definition used to map the sequence reads to a genomic region from which the sequence reads arose.

### Markov Models.

While the methods described above in conjunctions with FIGS. 2A and 2B are useful for a wide range of genomic regions that have incurred repeat expansions, in some embodiments the alignment module 101 uses different techniques for genomic regions that have incurred repeat expansions that are not readily described by a repeat definition 120. To this end, and referring to block 4400 of FIG. 3A as well as FIG. 1, in some embodiments, methods for mapping a plurality of sequence reads to a genomic region are provided that make use of a computer system comprising one or more processors and a system memory that encode an initial Markov model 126.

Referring to block 4402, in some embodiments, the genomic region that has incurred the repeat expansion has a length of between 200 and 5000 residues, between 1000 and 8000 residues, or between 2000 and 10,000 residues.

Referring to block 4404, as was in the case of the method disclosed above in conjunction with FIGS. 2A and 2B, in some embodiments, the methods comprise obtaining, in electronic form, a plurality of sequence reads that map to the genomic region.

Referring to block 4406, in some embodiments, the plurality of sequence reads have a mean length of at least 500, 600, 700, 800, 900, 1000, 1100, 1200, 1300, 1400, 1500, 1600, or 2000 residues. In some embodiments, the plurality of sequence reads have a mean, median or average length of about 5,000 bp to 50,000 bp long (e.g., about 5,000 bp, about 7,500 bp, about 10,000 bp, about 12,500 bp, about 15,000 bp, about 20,000 bp, about 25,000 bp, about 30,000 bp, about 35,000 bp, about 40,000 bp, about 45,000 bp, about 50,000 bp, about 55,000, about 60,000, about 65,000, about 70,000, about 75,000, or about 80,000). In some embodiments, the plurality of sequence reads have a mean, median, or average length of about 1000 bp, 2000 bp, 5000 bp, 10,000 bp, 50,000 bp or more.

Referring to block 4408, in some embodiments, the plurality of sequence reads comprises 1000, 2000, 5000, or 10,000 sequence reads. In some embodiments, the plurality of sequence reads comprises at least 1000, at least 2000, at least 3000, at least 4000, at least 5000, at least 6000, at least 7000, at least 8000, at least 9000, at least 10,000, at least 50,000, at least 100,000, at least 500,000, at least 1 million, at least 2 million, at least 3 million, at least 4 million, at least 5 million, at least 6 million, at least 7 million, at least 8 million, at least 9 million, or more sequence reads. In some embodiments, the plurality of sequence reads comprises at least 1×10⁷, at least 2×10⁷, at least 3×10⁷, at least 4×10⁷, at least 5×10⁷, at least 6×10⁷, at least 7×10⁷, at least 8×10⁷, at least 9×10⁷, at least 1×10⁸, at least 2×10⁸, at least 3×10⁸, at least 4×10⁸, at least 5×10⁸, at least 6×10⁸, at least 7×10⁸, at least 8×10⁸, at least 9×10⁸, at least 1×10⁹, or more sequence reads. In some embodiments, the plurality of sequence reads consists of no more than 5×10⁷, no more than 1×10⁷, no more than 5×10⁶, no more than 4×10⁶, no more than 3×10⁶, no more than 2×10⁶, no more than 1×10⁶, no more than 500,000, no more than 100,000, no more than 50,000, no more than 30,000, no more than 20,000, no more than 10,000, no more than 9000, no more than 8000, no more than 7000, no more than 6000, no more than 5000, no more than 4000, no more than 3000, no more than 2000, no more than 1000, or less sequence reads.

In some embodiments, plurality of sequence reads is obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

Referring to block 4410, in some embodiments, the plurality of sequence reads is generated in a single molecule sequencing-by-synthesis reaction. Referring to block 4412, in some embodiments, the single molecule sequencing by synthesis reaction is a Single Molecule, Real-Time (SMRT) Sequencing reaction. In some embodiments, the plurality of sequence reads is generated in a single molecule nanopore sequencing reaction. In some embodiments, the single molecule sequencing-by-synthesis reaction is sequencing of SMRTBELL© polynucleotide substrates in Single Molecule, Real-Time (SMRT©) sequencing from Pacific Biosciences, genomic fragments used in nanopore sequencing platforms, e.g., from Oxford Nanopore Technologies, Genia, and the like, or any other convenient single molecule sequencing platform. Examples of single molecule sequencing platforms and methods that can be used to produce sequence reads used by the systems and methods of the present disclosure, in some embodiments, are found in the following U.S. patents and U.S. patent application Publications, each of which is incorporated herein by reference: U.S. Pat. No. 8,324,914, US2013/0244340, US2015/0119259, US2010/0196203, US2011/0229877, US2016/0162634, U.S. Pat. No. 7,315,019, US2009/0087850, and US2018/0023134.

Referring to block 4414, in some embodiments, the methods comprise obtaining an initial Markov model for the genomic region. In a Markov model, transition probabilities between states for a Hidden Markov Model (HMM) can be determined using the nucleic acid distribution at each position in a set of sequence reads, thereby training the HMM. Hidden Markov models are described, for example, in Schliep et al., 2003, Bioinformatics 19(1):i255-i263, which is hereby incorporated by reference.

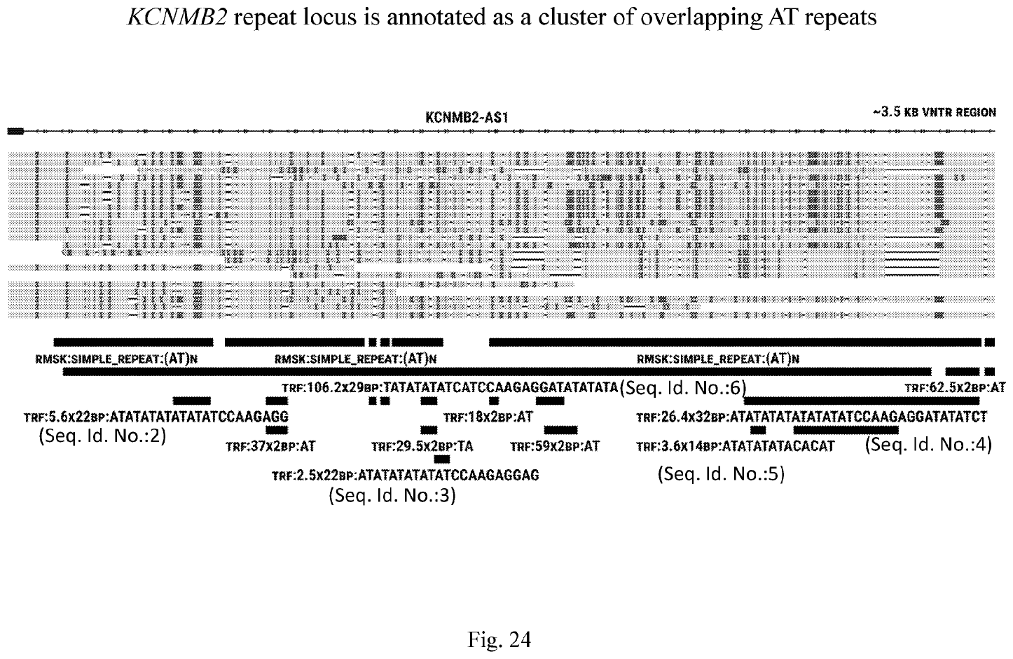

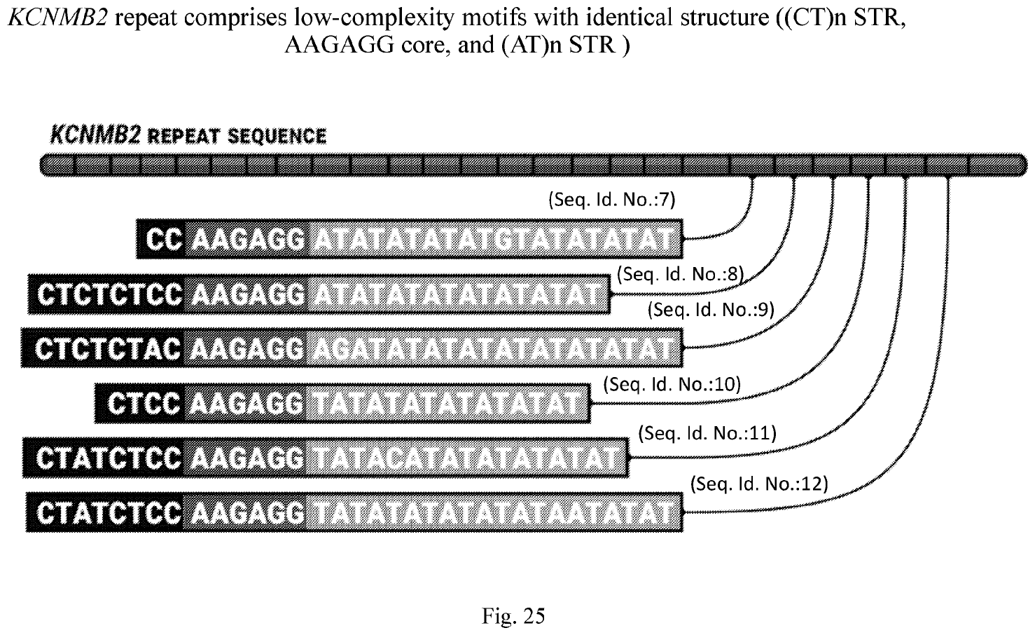

In some embodiments the regions that are known to incur repeat expansions require more sophisticated Markov models. For instance, FIG. 24 illustrates example sequence reads that have been aligned by a conventional mapping tool onto the KCNMB2 repeat locus. The KCNMB2 repeat locus is a notoriously difficult region to map sequence reads into, as illustrated by the overlapping and internally consistent reference annotations for this region shown for the KCNMB2 repeat locus at the bottom of FIG. 24. As illustrated in FIG. 25, the KCNMB2 repeat locus comprises low complexity motifs with identical structure ((CT)nSTR, AAGAG core and (AT)nSTR, where each n is the same or different and are each a positive integer. However, unlike the genomic situations illustrated for FIGS. 2A and 2B above, the repeat regions are not perfect. For instance, in the (CT)n region, there are sequences other than CT, such as CC and AC, and in the (AT)n region, there are sequences other than AT, such as AC and AAT.

To address genomic regions that have incurred complex repeat expansions such as the KCNMB2 repeat locus illustrated in FIGS. 24 and 25, one aspect of the present disclosure provides an initial Markov model 124 for the genomic region that comprises a plurality of states with a plurality of transition properties encoding at least (i) a first repeat for a first repeat region, (ii) a second repeat for a second repeat region, and (iii) an intermediate region linking the first repeat to the second repeat. In some embodiments a sequence read in the plurality of sequence reads has at least 2, 3, 4, 5, 6, 7, 9, 10, 11, 12, 13, 14, 15, 16, 17, 18, 19, or 20 repeats of the first repeat sequence followed by the fixed interrupt sequence followed by at least 2, 3, 4, 5, 6, 7, 9, 10, 11, 12, 13, 14, 15, 16, 17, 18, 19, or 20 repeats of the second repeat sequence. In some embodiments at least 5 sequence reads, at least 10 sequence reads, at least 15 sequence reads, at least 20 sequence reads, at least 50 sequence reads, at least 100 sequence reads, at least 250 sequence reads, at least 500 sequence reads, at least 1000 sequence reads, at least 5000 sequence reads in the plurality of sequence reads have at least 2, 3, 4, 5, 6, 7, 9, 10, 11, 12, 13, 14, 15, 16, 17, 18, 19, or 20 repeats of the first repeat sequence followed by the fixed interrupt sequence followed by at least 2, 3, 4, 5, 6, 7, 9, 10, 11, 12, 13, 14, 15, 16, 17, 18, 19, or 20 repeats of the second repeat sequence.

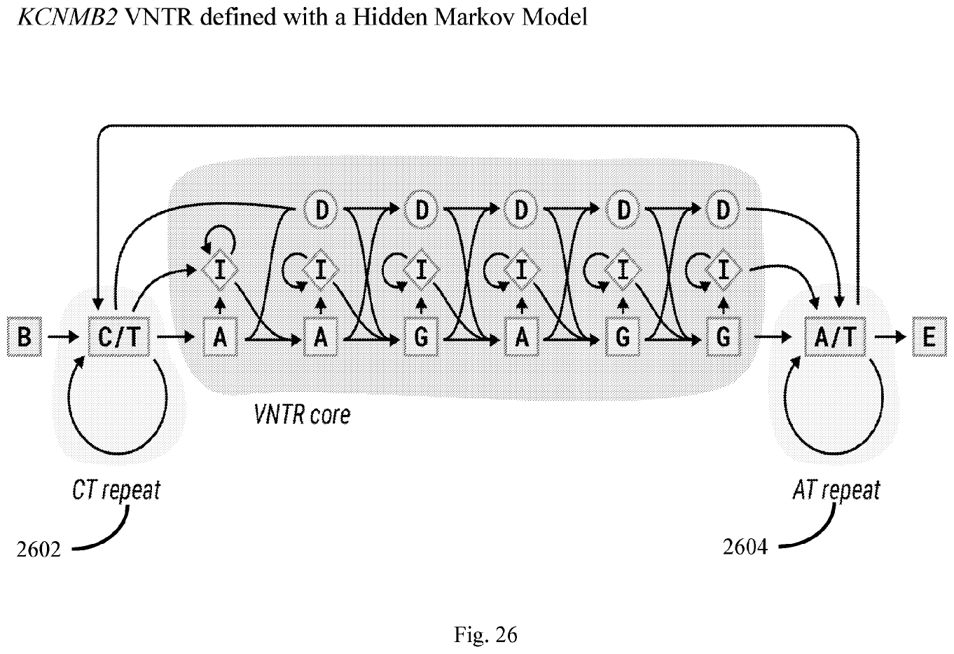

FIG. 26 illustrates. In FIG. 26, the CT repeat constitutes the first repeat for the first repeat region (CT)n in the example of FIG. 26, the AT repeat constitutes the second repeat for the second repeat region (AT)n in the example of FIG. 26, and the VNTR core constitutes the intermediate region linking the first repat to the second repeat. In the model, arrow 2602 will contain the probability, given a C/T that it is repeated in the CT repeat region, the VNTR core will encode a number of probabilities across the core to accommodate all the possible sequences in the plurality of sequences, while arrow 2604 will contain the probability, given an A/T that it is repeated in the AT repeat. The plurality of sequences can be aligned on the AAGAGG core, as illustrated in FIG. 25, and the aligned sequences can them be used to train the transition probabilities (e.g., transitions 2602 and 2604) of the Markov model of FIG. 26.

Referring to block 4418, in some embodiments, the first region further comprises one or more residues that are other than the first repeat sequence, and the second region further comprises one or more residues that are other than the second repeat sequence. Thus, while FIG. 26 illustrates one possible Markov model that can be used for the KCNMB2 repeat locus, the model is shown by way of example to illustrate the important features of the model, such as at least two repeat transition probabilities for two different repeat regions (arrows 2602 and 2604). However, in practice, more complex Markov models that encode for more rare states such as, for instance, in the (CT)ₙ region, encoding the sequences other than CT, such as CC and AC as states within the (CT)ₙ portion of the Markov model with requisite transition probabilities, and in the (AT)ₙ region, encoding sequences other than AT, such as AC and AAT as states within the (AT)ₙ portion of the Markov model with requisite transition probabilities.

Referring to block 4416, in some embodiments, the first region comprises one or more instances of a first repeat sequence having a length of between 2 and 100 residues, the intermediate regions has a length of between 2 and 100 residues, and the second region comprises one or more instances of second repeat sequence having has a length of between 2 and 100 residues.

Referring to block 4420, in some embodiments, the methods comprise refining the initial Markov model using the plurality of sequence reads, thereby obtaining a refined Markov model. For instance, as discussed above, the sequence reads mapping to KCNMB2 can be aligned against the AAGAGG core and then used to train the transition probabilities of the Markov model illustrated in FIG. 26.

Referring to block 4420, in some embodiments, the methods comprise, for each respective sequence read in the plurality of sequences, performing a procedure comprising (i) using the respective sequence read to find a highest probability path through the Markov model, and (ii) using the highest probability path to map the respective sequence read to the genomic region. Thus, with the Markov model now trained, the sequence 104 of each respective sequence read 106 is run through the Markov model to obtain the highest probability path through the Markov model for the respective sequence read 106. This highest probability path represents the segmentation for the respective sequence read, which, as in the case of the methods described above in conjunction with FIGS. 2A and 2B. is then used to map the sequence read to the genomic region.

Referring to block 4422, in some embodiments, the using the highest probability path to map the respective sequence read to the genomic region comprises producing a respective plurality of segmentations that are each a permutation of the highest probability path, selecting a respective first segmentation in the respective plurality of segmentations having a best score as the segmentation for the respective sequence read, and using the respective first segmentation to map the respective sequence read to the genomic region. While the highest probability path through the refined Markov model 126 reduces, by orders of magnitude, the astronomical number of possible segmentations that the brute force approach considers, it is still the case that optimization of the segmentation given by the highest probable path is needed, results in the need to evaluate 100, 500, 1000, 2000, 3000, 4000, 5000, 10,000, 100,000, 1×10⁶, or more different segmentations for each respective sequence read in the plurality of sequence reads based on the respective highest probable path through the trained Markov model for each such sequence read. Each such computation requires a scoring of the sequence 106 of the sequence read 104 to the sequence of the candidate segmentation to find the best score. Each such comparison requires matching the sequence 106 of the sequence read to the sequence of the candidate sequence. In some embodiments, the segmentation of the highest probable path with deletions, insertions and gaps introduced are also considered in order to map the sequence read to the genomic region, adding still more complexity to the mapping. Thus, referring to block 4424, in some embodiments, the respective plurality of segmentations comprises 100, 500, 1000, 2000, 3000, 4000, 5000, 10,000, 100,000 or 1×10⁶ different segmentations for reach respective sequence read in the plurality of sequence reads. This is further in the context that typical practical applications require 10, 100, 500 1000, 2000, 5000, 10,000, or more sequence reads mapping to a particular genomic region.

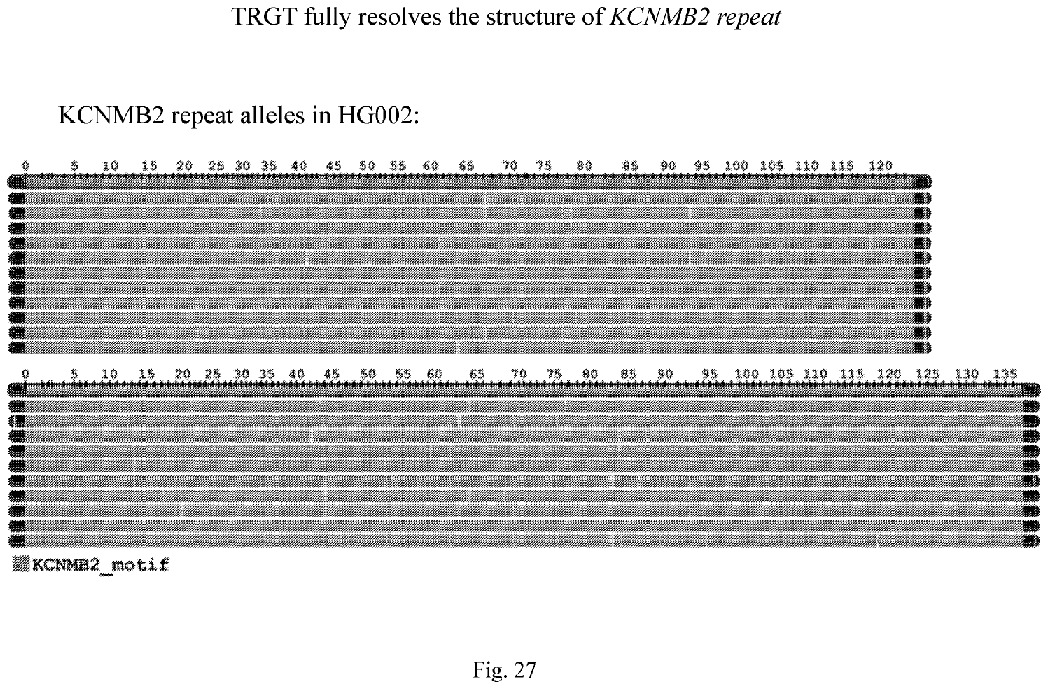

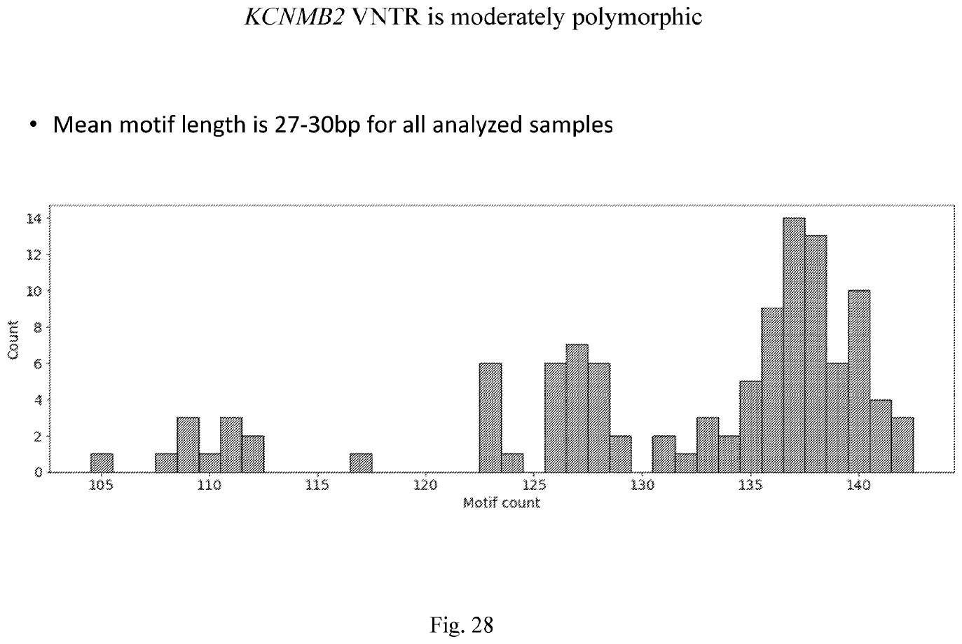

FIG. 27 illustrates the improvement that the disclosed methods achieve in mapping sequences to KCNMB2 in accordance with FIG. 3 over the conventional mapping of FIG. 24 for the same sequence reads used in FIG. 24. FIG. 28 provides an analysis of the mapped sequences.

In some embodiments, the genotyping SNP is used to resolve some of the repeats that the Markov model was unable to satisfactorily resolve using the techniques described above in conjunction with block 4322.

### EXAMPLES

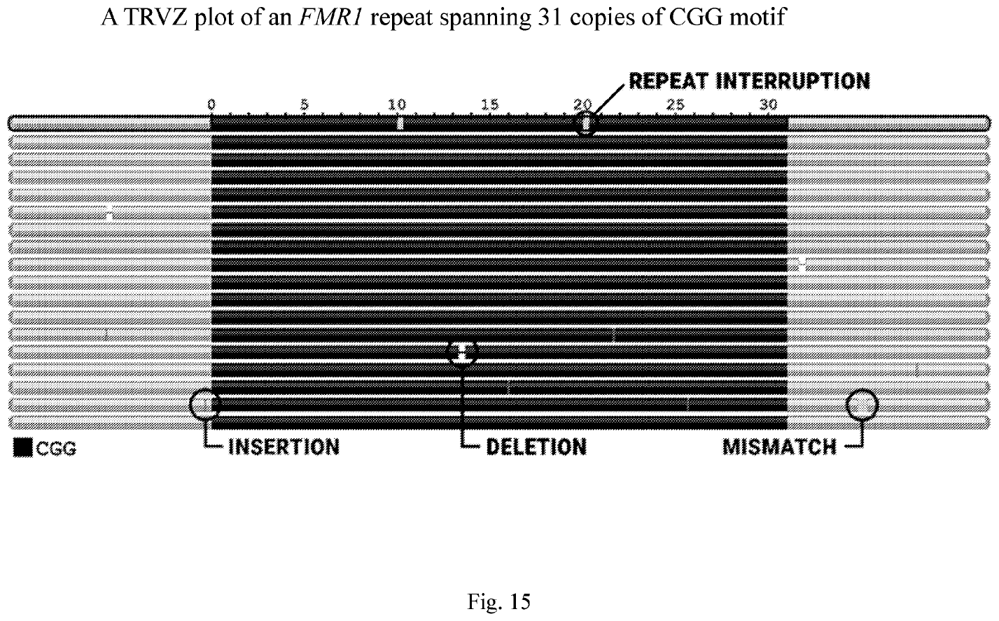

Example 1. FIG. 15 illustrates a lineup plot of sequence reads mapping to a genomic location that includes a portion of the FMR1 expansion in accordance with a FMR1 repeat definition (CAG)nCAACAG(CCG)n, in accordance with the method disclosed in FIGS. 2A and 2B, in which sequence reads have been successfully mapped to the genome even though the genome includes 31 contiguous copies of the CGG motif.

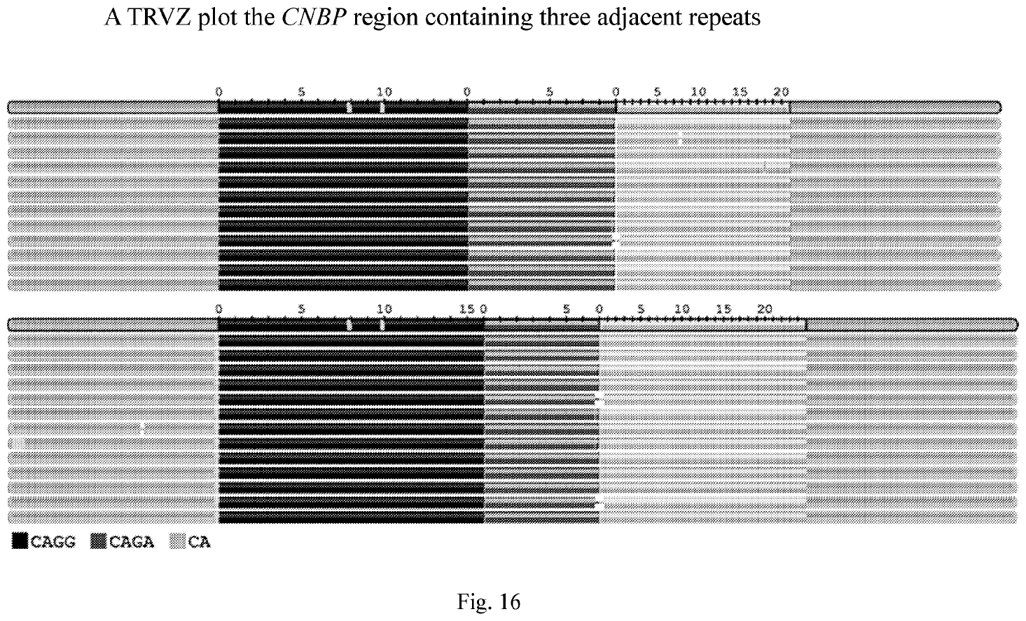

Example 2. FIG. 16 illustrates a lineup plot of sequence reads mapping to a genomic location that includes the CNBP expansion in accordance with a CNBP repeat definition that includes three different adjacent repeats CAGG, CAGA, and CA, in accordance with the method disclosed in FIGS. 2A and 2B.

Example 3. FIG. 17 illustrates how the method of FIGS. 2A and 2B is sufficiently powerful to map sequence reads to a genomic region having repeats even when the repeat definition 120 fails to include a motif that is present in the genomic region. In FIG. 17, the method of FIGS. 2A and 2B has been used to successfully map sequence reads to the RFC1 genomic region for a subject that includes a non-reference AAGAG motif. That is, the AAGAG motif is not in the repeat definition 120 for RFC1.

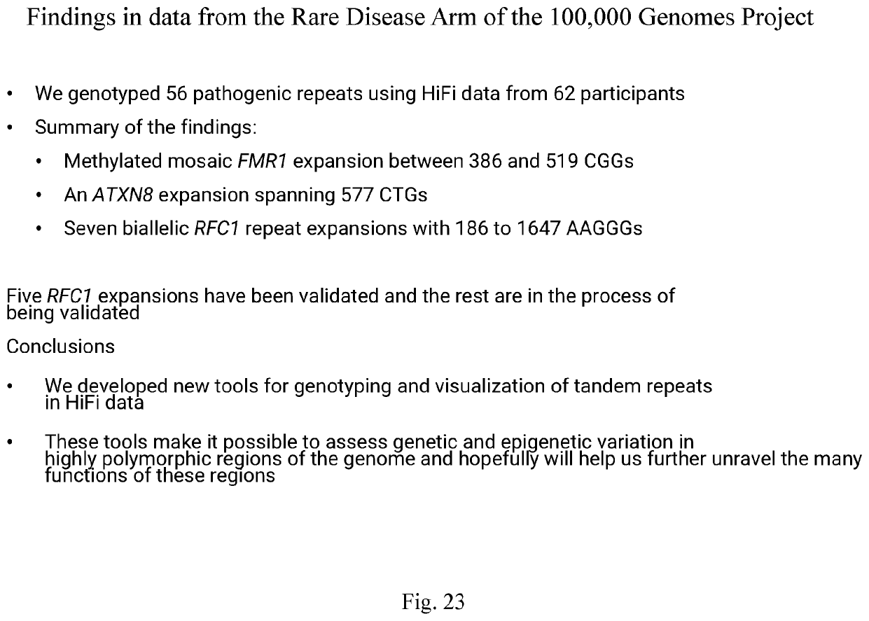

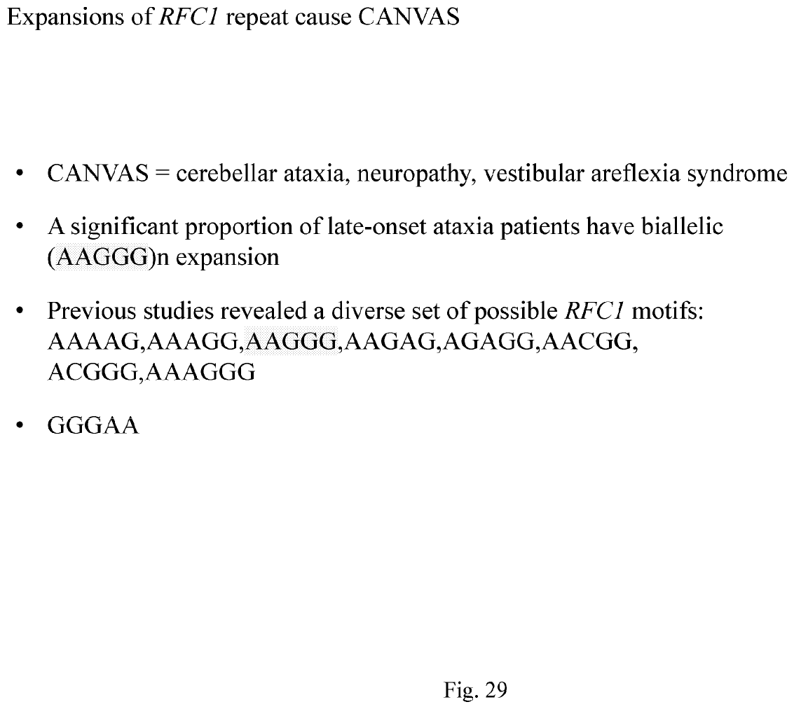

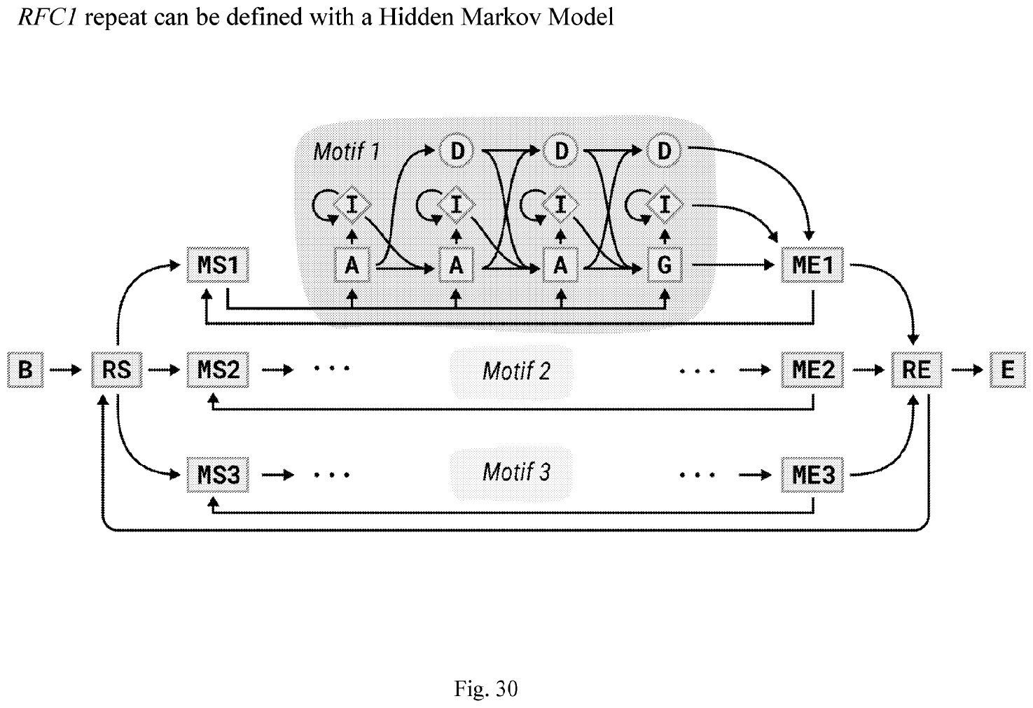

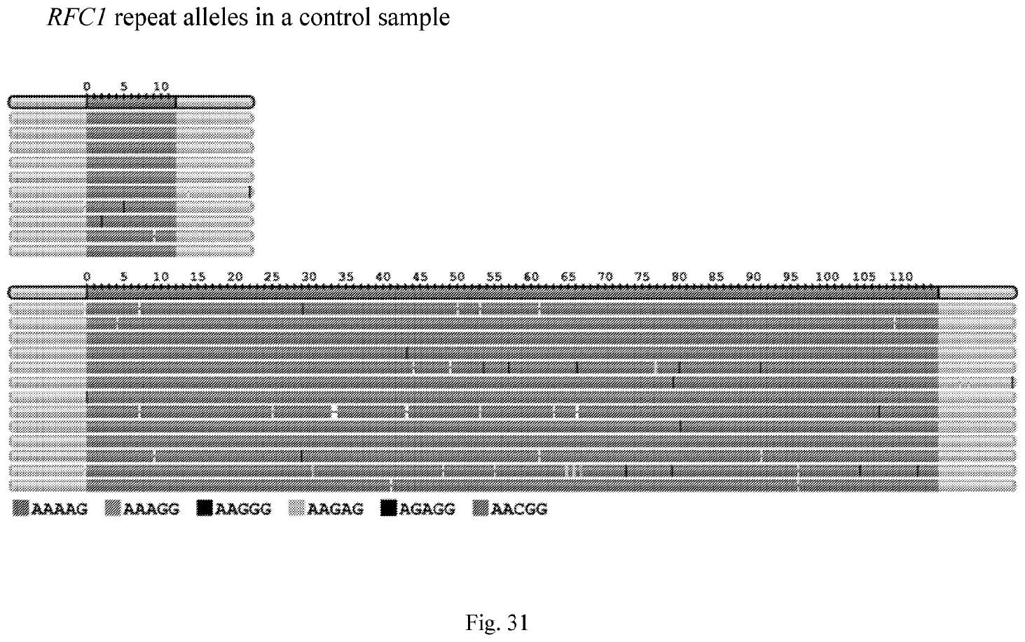

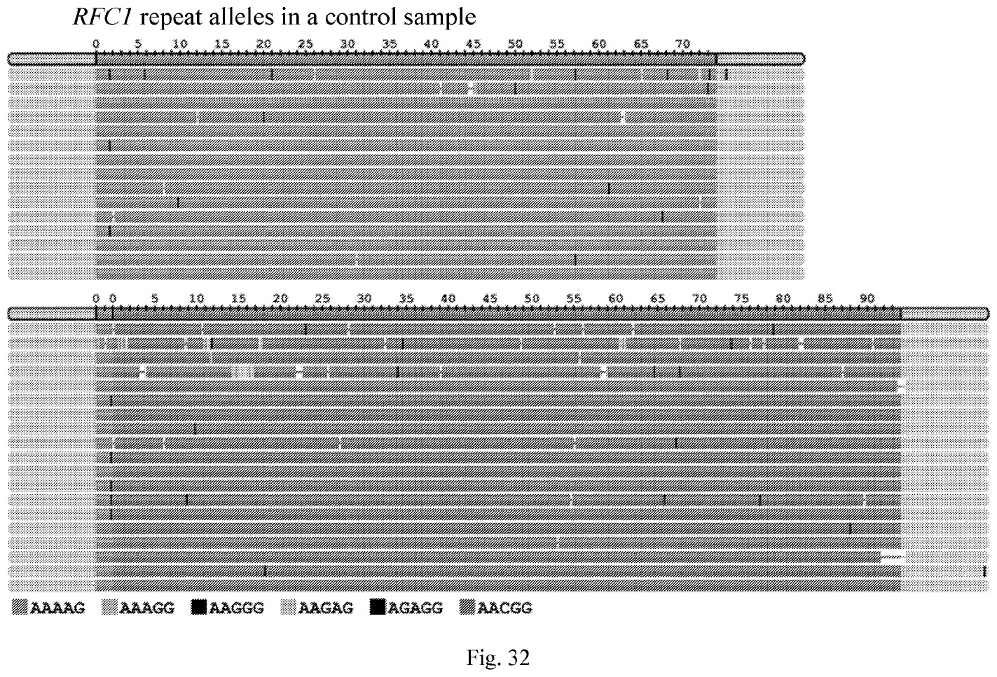

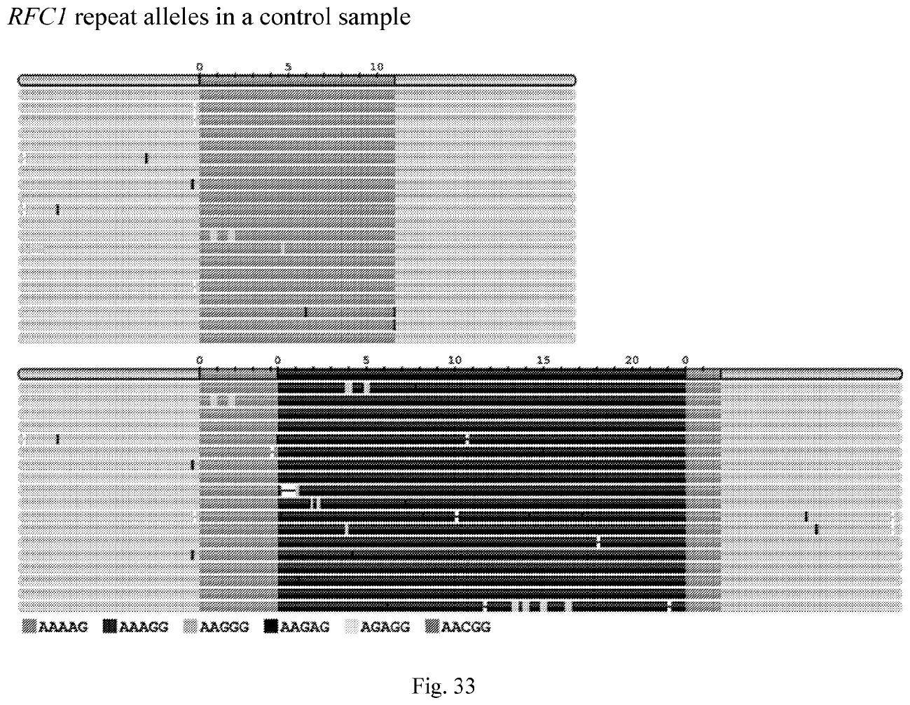

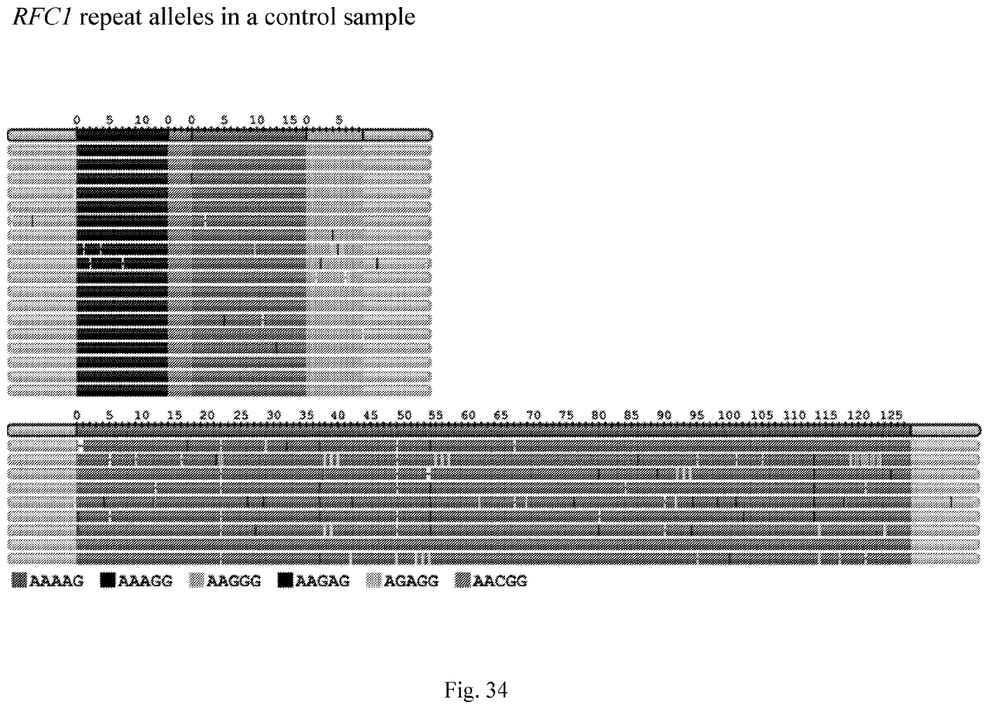

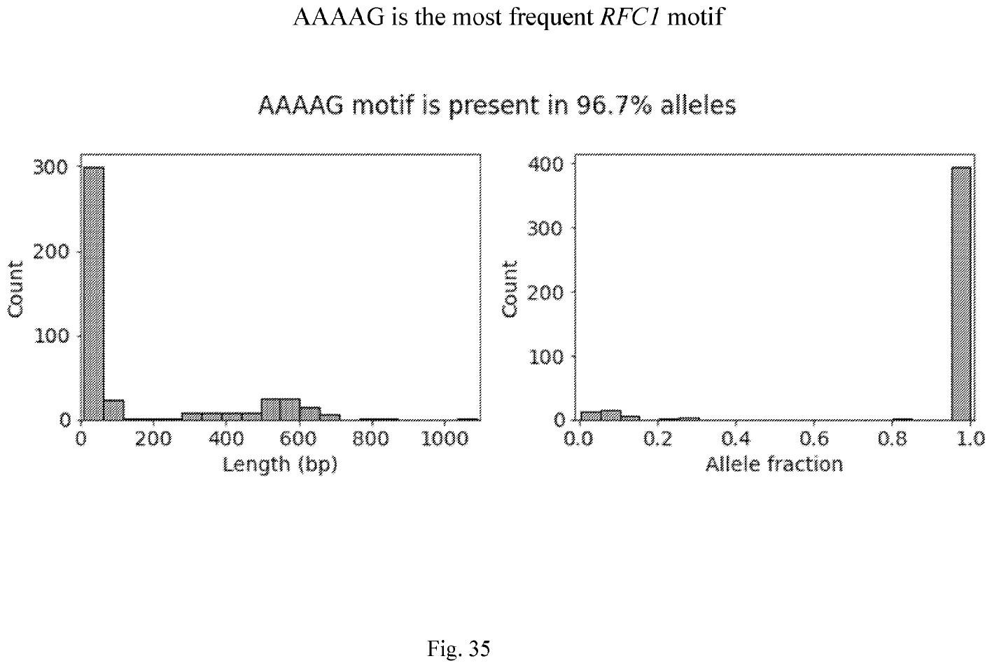

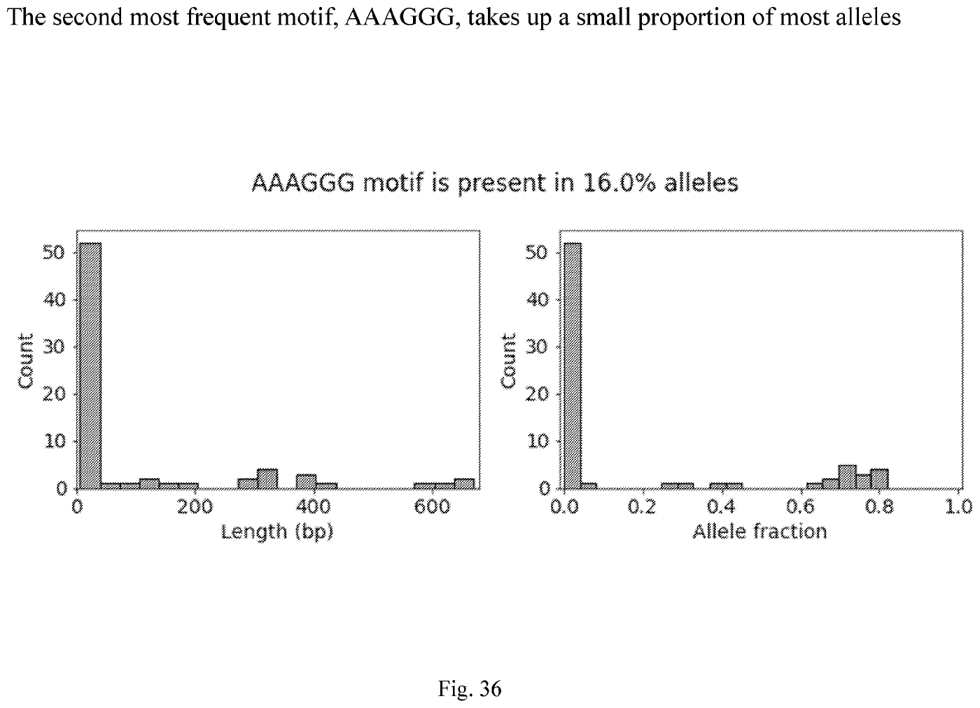

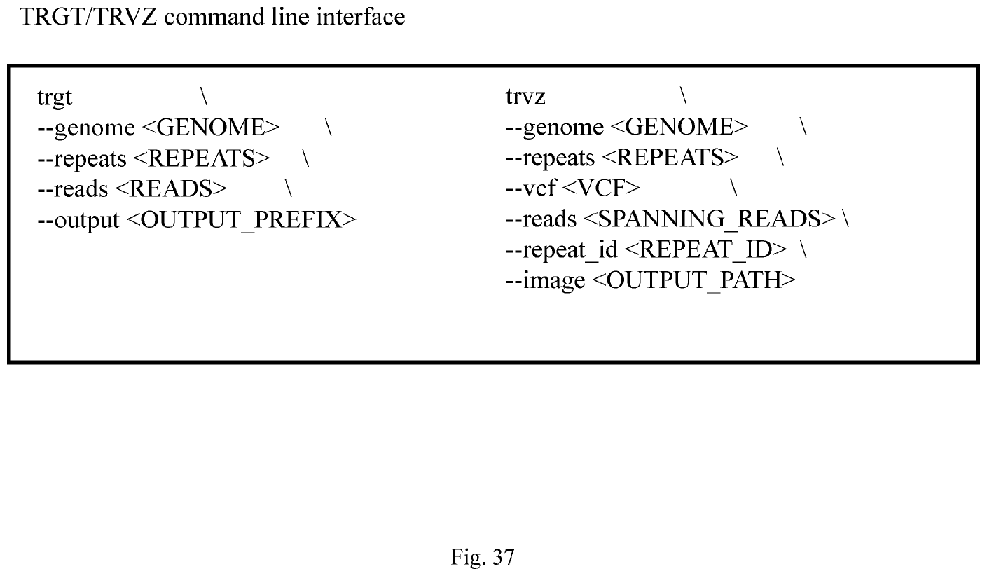

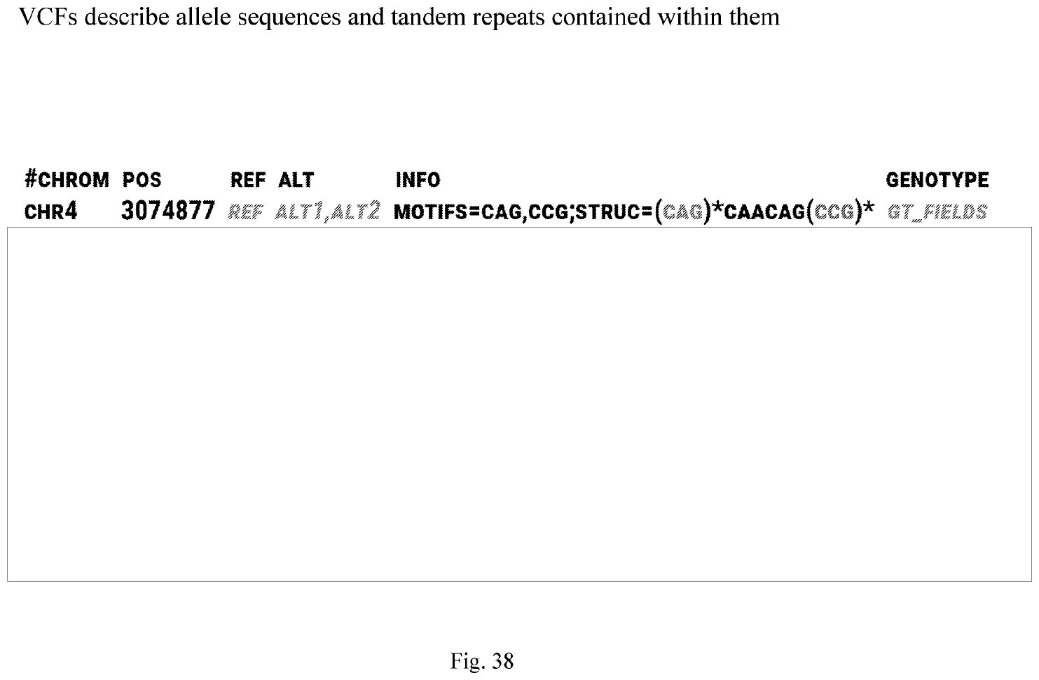

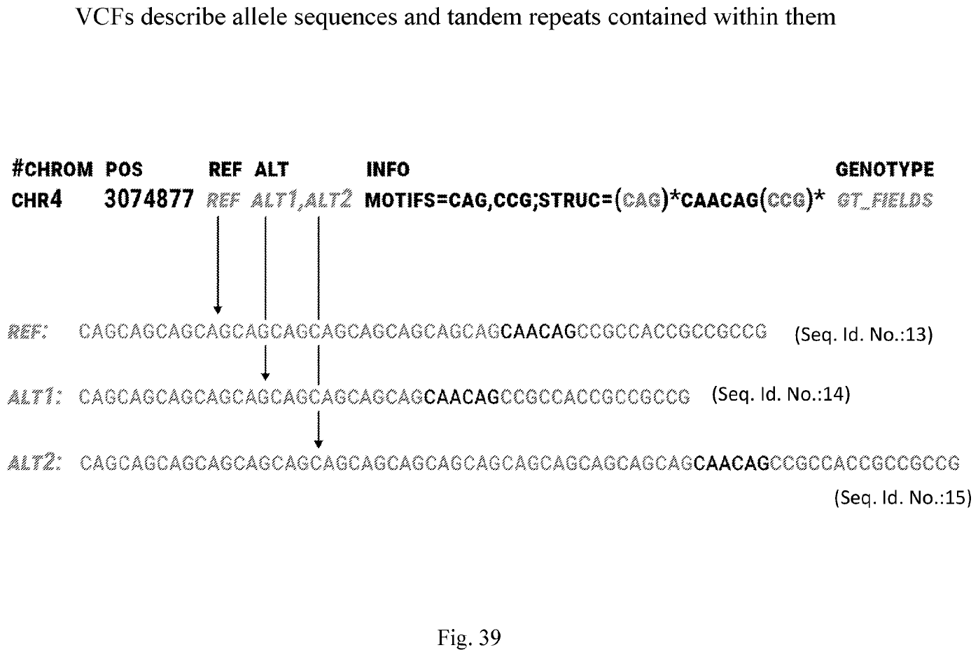

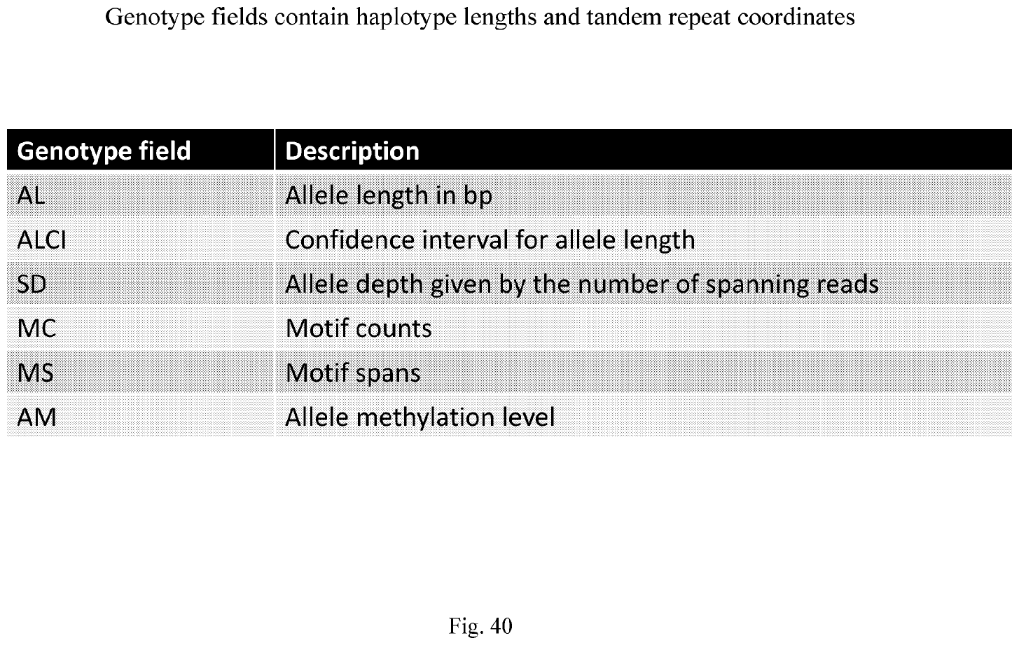

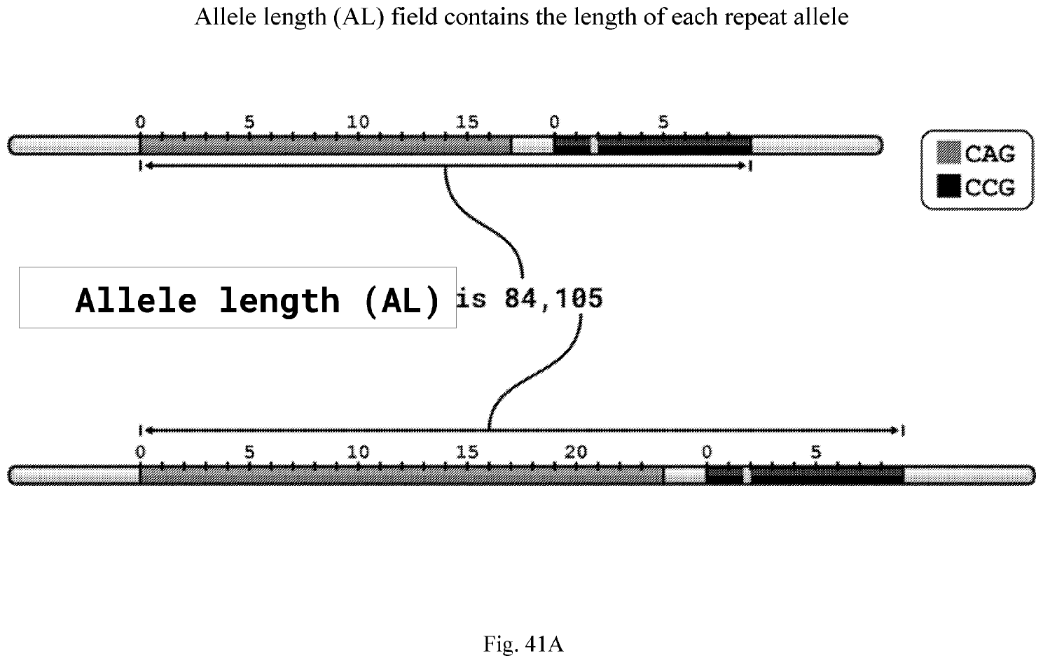

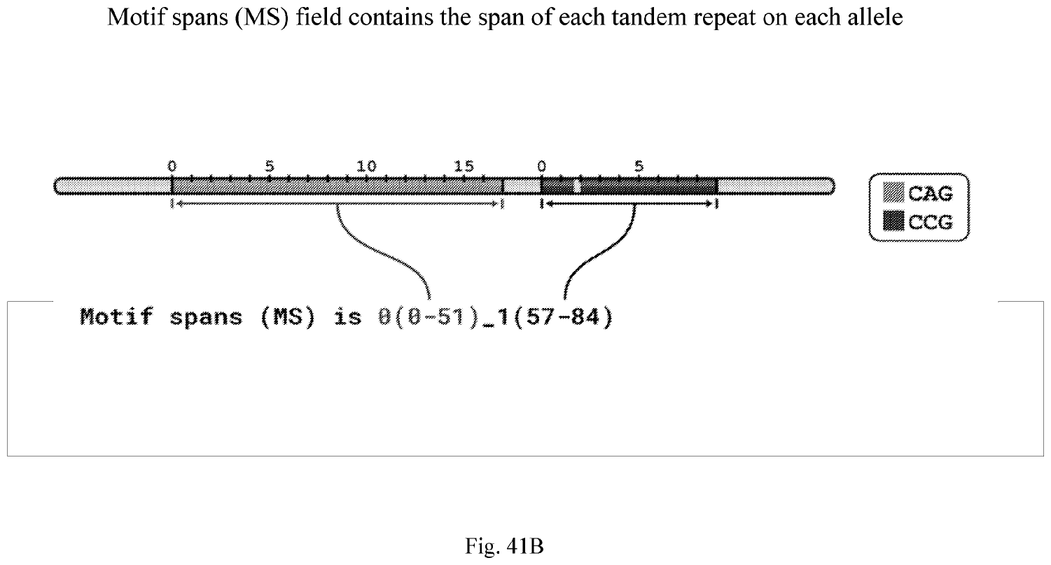

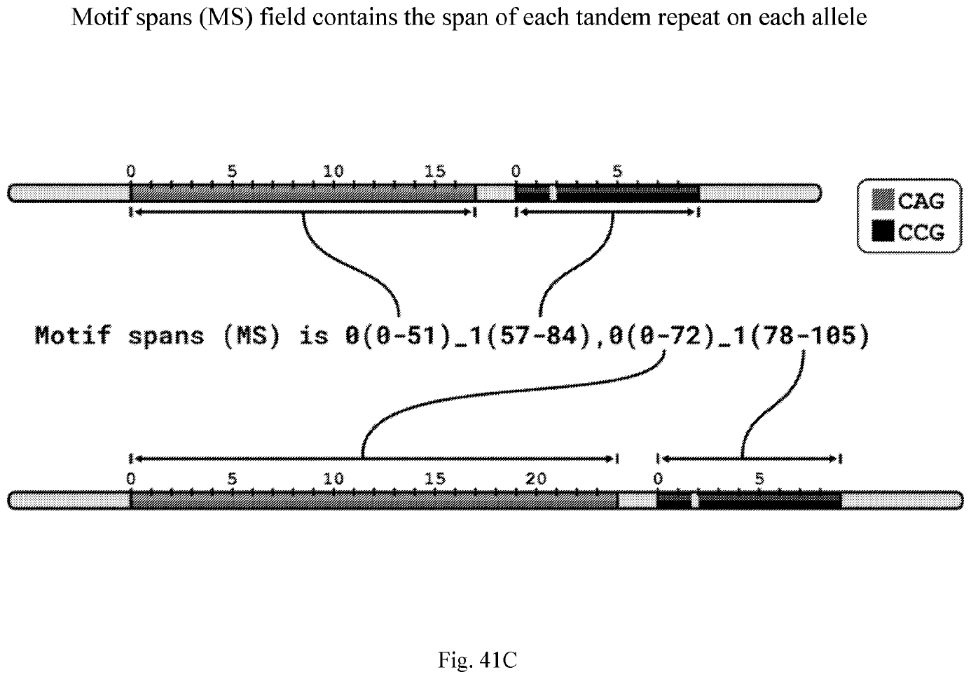

Example 4. FIG. 29 illustrates details of another genomic region that undergoes repeat expansion that is suitable for the mapping methods described above in conjunction with FIG. 3. The genomic region encodes RFC1, which has been associated with cerebellar ataxia, neuropathy, vestibular areflexia syndrome (CANVAS). Previous studies revealed a diverse set of possible RFC1 motifs: AAAAG, AAAGG, AAGGG, AAGAG, AGAGG, AACGG, ACGGG, and AAAGGG, the expansion of one of which, (AAGGG)ₙ, has been associated with late-onset ataxia. FIG. 30 illustrates the Markov model that has been defined for genomic region in accordance with the methods described above in conjunction with FIG. 3. FIGS. 31, 32, 33, and 34 illustrate how the Markov model, using the methods described in FIG. 3, enable the mapping of a plurality of sequence reads from a control sample to RFC1. FIGS. 35 and 36 detail statistics of the genotypes represented by these mapped sequence reads. FIG. 37 illustrates a command line interface for the alignment and visualization tools of the present disclosure. FIGS. 38 and 39 illustrate how VCFs describe allele sequences and tandem repeats contained within them in accordance with an embodiment of the present disclosure. FIG. 40 illustrates how genotype fields contain haplotype lengths and tandem repeat coordinates in accordance with some embodiments of the present disclosure. FIG. 41A illustrates how the allele length (AL) field contains the length of each repeat allele in accordance with some embodiments of the present disclosure. FIGS. 41B and 41C illustrate how the motif spans (FS) field contains the span of each tandem repeat on each allele in accordance with some embodiments of the present disclosure. FIG. 23 illustrates how methylated mosaic FMR1 expansion between 386 and 519 CGGs, an ATUV8 expansion spanning 577 CTGs, and seven biallelic RFC1 repeat expansions with 186 to 1647 AAGGGs were discovered using the systems and methods of the present disclosure.

### REFERENCES CITED AND ALTERNATIVE EMBODIMENTS

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

The present invention can be implemented as a computer program product that comprises a computer program mechanism embedded in a nontransitory computer readable storage medium. For instance, the computer program product could contain the program modules shown in FIG. 1 and/or described in FIGS. 2A, 2B, 3A, and/or 3B. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed is:

1. A method for mapping a plurality of sequence reads to a genomic region, the method comprising:
at a computer system comprising one or more processors and a system memory:
a) obtaining, in electronic form, the plurality of sequence reads, wherein each sequence read in the plurality of sequence reads overlaps the genomic region;
b) obtaining a repeat definition for the genomic region, wherein the repeat region comprises at least (i) a first region comprising a first variable number of repeats of a first repeat sequence, (ii) a second region comprising a second variable number of repeats of a second repeat sequence, and (iii) a fixed interruption sequence between the first region and the second region;
c) for each respective sequence read in the plurality of sequences, performing a procedure comprising:
(i) using the repeat definition to generate a corresponding graph for the respective sequence read, the corresponding graph comprising a respective plurality of nodes and a respective plurality of edges, by scanning the respective sequence read from a first end to a second end for perfect matches to each motif in a corresponding plurality of motifs in the repeat definition, wherein
each node in the respective plurality of nodes represents a motif in the plurality of motifs,
the plurality of motifs comprises at least a first instance of the first repeat sequence, a first instance of the second repeat sequence, an instance of the fixed interruption sequence, and a second instance of the first or second repeat sequence,
each edge in the plurality of edge connects a corresponding node of a first motif and corresponding node of a second motif in the plurality of motifs observed to be contiguous in the respective sequence read, and
the corresponding graph has one or more branch points,
(ii) identifying a longest path through the respective graph as the candidate segmentation for the respective sequence read, and
(iii) using the longest path in the respective graph to map the respective sequence read to the genomic region.

2. The method of claim 1, wherein the repeat definition specifies that the first repeat sequence is repeated at least 1, 2, 3, 4, 5, 6, 7, 8, 9, or 10 times and that the second repeat sequence is repeated at least 1, 2, 3, 4, 5, 6, 7, 8, 9, or 10 times.

3. The method of claim 1, wherein the repeat definition specifies that the first repeat sequence is repeated at least 10, 20, 30, 40, 50, 60, 70, 80, 90, or 100 times and that the second repeat sequence is repeated at least 10, 20, 30, 40, 50, 60, 70, 80, 90, or 100 times.

4. The method of any one of claims 1-3, wherein the first repeat sequence has a length of between 2 and 100 residues, the fixed interruption sequence has a length of between 2 and 100 residues, and the second repeat sequence has a length of between 2 and 100 residues.

5. The method of any one of claims 1-4, wherein the plurality of sequence reads have a mean length of at least 500, 600, 700, 800, 900, 1000, 1100, 1200, 1300, 1400, 1500, 1600, or 2000 residues.

6. The method of any one of claims 1-5, wherein the plurality of sequence reads comprises 1000, 2000, 5000, or 10,000 sequence reads.

7. The method of any one of claims 1-6, wherein the using (iii) comprises:
producing a respective plurality of segmentations in accordance with the longest path and the repeat definition,
selecting a respective first segmentation in the respective plurality of segmentations having a best score as the segmentation for the respective sequence read, and
using the respective first segmentation to map the respective sequence read to the genomic region.

8. The method of claim 7, wherein the respective plurality of segmentations comprises 100, 500, 1000, 2000, 3000, 4000, 5000, 10,000, 100,000 or 1×10⁶ different segmentations.

9. The method of any one of claims 1 to 6, wherein the plurality of sequence reads are generated in a single molecule sequencing-by-synthesis reaction.

10. The method of claim 9, wherein the single molecule sequencing by synthesis reaction is a Single Molecule, Real-Time (SMRT) Sequencing reaction.

11. The method of any one of claims 1-10 wherein the genomic region is in a genome.

12. The method of claim 11, wherein the genome is a human genome.

13. The method of claim 12, wherein the plurality of sequence reads originate from a subject, the genomic region is associated with a disease and the using the longest path in the respective graph to map the respective sequence read to the genomic region identifies a status, stage, presence, or absence of the disease in the subject.

14. The method of claim 13 wherein the disease is a tandem repeat disorder, Alzheimer's, an autism spectrum disorder, Fragile X syndrome, epilepsy, amyotrophic lateral sclerosis, Huntington's disease, Kennedy's disease, myotonic dystrophy, or a spinocerebellar ataxia.

15. The method of any one of claims 1-14, wherein the obtaining the repeat definition for the genomic region comprises identifying the repeat definition from among a plurality of repeat definitions based on an identity of the genomic region.

16. The method of claim 15, wherein the plurality of repeat definitions comprises 10 or more repeat definitions, 100 or more repeat definitions, 1000 or more repeat definitions, 100,000 or more repeat definitions, or 1×10⁶ or more repeat definitions.

17. The method of any one of claims 1-14, wherein the plurality of sequence reads originate from a subject and the method further comprises using the mapping of the plurality of sequence reads to phase the genomic region.

18. The method of any one of claims 1-14, wherein the plurality of sequence reads originate from a subject and the method further comprises using the mapping of the plurality of sequence reads to determine a status of a genetic disease associated with the genomic region in the subject.

19. A method, for mapping a plurality of sequence reads to a genomic region, the method comprising:
at a computer system comprising one or more processors and a system memory:
a) obtaining, in electronic form, the plurality of sequence reads, wherein each sequence read in the plurality of sequence reads overlaps the genomic region;
b) obtaining an initial Markov model for the genomic region, wherein the initial Markov model comprises at least (i) a first repeat for a first repeat region, (ii) a second repeat for a second repeat region, and (iii) an intermediate region linking the first repeat to the second repeat;
c) refining the initial Markov model using the plurality of sequence reads, thereby obtaining a refined Markov model; and
d) for each respective sequence read in the plurality of sequences, performing a procedure comprising:
(i) using the respective sequence read to find a highest probability path through the Markov model, and
(ii) using the highest probability path to map the respective sequence read to the genomic region.

20. The method of claim 19, wherein the first region comprises one or more instances of a first repeat sequence having a length of between 2 and 100 residues, the intermediate regions has a length of between 2 and 100 residues, and the second region comprises one or more instances of second repeat sequence having has a length of between 2 and 100 residues.

21. The method of claim 20, wherein
the first region further comprises one or more residues that are other than the first repeat sequence, and
the second region further comprises one or more residues that are other than the second repeat sequence.

22. The method of any one of claims 19-21, wherein the genomic region has a length of between 200 and 5000 residues.

23. The method of any one of claims 19-21, wherein the genomic region has a length of between 1000 and 8000 residues.

24. The method of any one of claims 19-21, wherein the genomic region has a length of between 2000 and 10,000 residues.

25. The method of any one of claims 19-24, wherein the plurality of sequence reads have a mean length of at least 500, 600, 700, 800, 900, 1000, 1100, 1200, 1300, 1400, 1500, 1600, or 2000 residues.

26. The method of any one of claims 19-25, wherein the plurality of sequence reads comprises 1000, 2000, 5000, or 10,000 sequence reads.

27. The method of any one of claims 19-26, wherein the using (ii) comprises:
producing a respective plurality of segmentations that are each a permutation of the highest probability path,
selecting a respective first segmentation in the respective plurality of segmentations having a best score as the segmentation for the respective sequence read, and
using the respective first segmentation to map the respective sequence read to the genomic region.

28. The method of claim 27, wherein the respective plurality of segmentations comprises 100, 500, 1000, 2000, 3000, 4000, 5000, 10,000, 100,000 or 1×10⁶ different segmentations.

29. The method of any one of claims 19 to 28, wherein the plurality of sequence reads is generated in a single molecule sequencing-by-synthesis reaction.

30. The method of claim 29, wherein the single molecule sequencing by synthesis reaction is a Single Molecule, Real-Time (SMRT) Sequencing reaction.

31. The method of any one of claims 19-30, wherein the genomic region is in a genome.

32. The method of claim 19, wherein the genome is a human genome.

33. The method of claim 32, wherein the plurality of sequence reads originate from a subject, the genomic region is associated with a disease and the using the highest probability path to map the respective sequence read to the genomic region identifies a status of the disease in the subject.

34. The method of claim 33, wherein the disease is Alzheimer's, autism, epilepsy, or ALS.

35. The method of any one of claims 19-34, wherein the obtaining the repeat definition for the genomic region comprises identifying the repeat definition from among a plurality of repeat definitions based on an identity of the genomic region.

36. The method of claim 35, wherein the plurality of repeat definitions comprises 10 or more repeat definitions, 100 or more repeat definitions, 1000 or more repeat definitions, 100,000 or more repeat definitions, or 1×10⁶ or more repeat definitions.

37. The method of any one of claims 19-33, wherein the plurality of sequence reads originate from a subject and the method further comprises using the highest probability path to map the respective sequence read to the genomic region to phase the genomic region.

38. The method of any one of claims 19-33, wherein the plurality of sequence reads originate from a subject and the method further comprises using the mapping of the plurality of sequence reads to determine a status of a genetic disease associated with the genomic region in the subject.

39. A system for mapping a plurality of sequence reads to a genomic region, comprising:
a memory;
input/output; and
a processor coupled to the memory, wherein the system is configured to perform a method comprising:
a) obtaining, in electronic form, the plurality of sequence reads, wherein each sequence read in the plurality of sequence reads overlaps the genomic region;
b) obtaining a repeat definition for the genomic region, wherein the repeat region comprises at least (i) a first region comprising a first variable number of repeats of a first repeat sequence, (ii) a second region comprising a second variable number of repeats of a second repeat sequence, and (iii) a fixed interruption sequence between the first region and the second region;
c) for each respective sequence read in the plurality of sequences, performing a procedure comprising:
(i) using the repeat definition to generate a corresponding graph for the respective sequence read, the corresponding graph comprising a respective plurality of nodes and a respective plurality of edges, by scanning the respective sequence read from a first end to a second end for perfect matches to each motif in a corresponding plurality of motifs in the repeat definition, wherein
each node in the respective plurality of nodes represents a motif in the plurality of motifs,
the plurality of motifs comprises at least a first instance of the first repeat sequence, a first instance of the second repeat sequence, an instance of the fixed interruption sequence, and a second instance of the first or second repeat sequence,
each edge in the plurality of edge connects a corresponding node of a first motif and corresponding node of a second motif in the plurality of motifs observed to be contiguous in the respective sequence read, and
the corresponding graph has one or more branch points,
(ii) identifying a longest path through the respective graph as the candidate segmentation for the respective sequence read, and
(iii) using the longest path in the respective graph to map the respective sequence read to the genomic region.

40. A non-transitory computer readable storage medium, wherein the non-transitory computer readable storage medium stores instructions, which when executed by a computer system, cause the computer system to perform a method for mapping a plurality of sequence reads to a genomic region, the method comprising:
a) obtaining, in electronic form, the plurality of sequence reads, wherein each sequence read in the plurality of sequence reads overlaps the genomic region;
b) obtaining a repeat definition for the genomic region, wherein the repeat region comprises at least (i) a first region comprising a first variable number of repeats of a first repeat sequence, (ii) a second region comprising a second variable number of repeats of a second repeat sequence, and (iii) a fixed interruption sequence between the first region and the second region;
c) for each respective sequence read in the plurality of sequences, performing a procedure comprising:
(i) using the repeat definition to generate a corresponding graph for the respective sequence read, the corresponding graph comprising a respective plurality of nodes and a respective plurality of edges, by scanning the respective sequence read from a first end to a second end for perfect matches to each motif in a corresponding plurality of motifs in the repeat definition, wherein
each node in the respective plurality of nodes represents a motif in the plurality of motifs,
the plurality of motifs comprises at least a first instance of the first repeat sequence, a first instance of the second repeat sequence, an instance of the fixed interruption sequence, and a second instance of the first or second repeat sequence,
each edge in the plurality of edge connects a corresponding node of a first motif and corresponding node of a second motif in the plurality of motifs observed to be contiguous in the respective sequence read, and
the corresponding graph has one or more branch points,
(ii) identifying a longest path through the respective graph as the candidate segmentation for the respective sequence read, and
(iii) using the longest path in the respective graph to map the respective sequence read to the genomic region.

41. A system for mapping a plurality of sequence reads to a genomic region, comprising:
a memory;
input/output; and
a processor coupled to the memory, wherein the system is configured to perform a method comprising:
a) obtaining, in electronic form, the plurality of sequence reads, wherein each sequence read in the plurality of sequence reads overlaps the genomic region;
b) obtaining an initial Markov model for the genomic region, wherein the initial Markov model comprises at least (i) a first repeat for a first repeat region, (ii) a second repeat for a second repeat region, and (iii) an intermediate region linking the first repeat to the second repeat;
c) refining the initial Markov model using the plurality of sequence reads, thereby obtaining a refined Markov model; and
d) for each respective sequence read in the plurality of sequences, performing a procedure comprising:
(i) using the respective sequence read to find a highest probability path through the Markov model, and
(ii) using the highest probability path to map the respective sequence read to the genomic region.

42. A non-transitory computer readable storage medium, wherein the non-transitory computer readable storage medium stores instructions, which when executed by a computer system, cause the computer system to perform a method for mapping a plurality of sequence reads to a genomic region, the method comprising:
a) obtaining, in electronic form, the plurality of sequence reads, wherein each sequence read in the plurality of sequence reads overlaps the genomic region;
b) obtaining an initial Markov model for the genomic region, wherein the initial Markov model comprises at least (i) a first repeat for a first repeat region, (ii) a second repeat for a second repeat region, and (iii) an intermediate region linking the first repeat to the second repeat;
c) refining the initial Markov model using the plurality of sequence reads, thereby obtaining a refined Markov model; and
d) for each respective sequence read in the plurality of sequences, performing a procedure comprising:
(i) using the respective sequence read to find a highest probability path through the Markov model, and
(ii) using the highest probability path to map the respective sequence read to the genomic region.


SEQUENCE LISTING (17 sequences)

SEQ ID NO: 1 - DNA, 39 bp, Homo sapiens
  source 1..39: mol_type = other DNA
cagcagcagcagccgcagcagcaacagccgccgcagccg

SEQ ID NO: 2 - DNA, 22 bp, Homo sapiens
  source 1..22: mol_type = other DNA
atatatatatatatccaagagg

SEQ ID NO: 3 - DNA, 22 bp, Homo sapiens
  source 1..22: mol_type = other DNA
atatatatatatccaagaggag

SEQ ID NO: 4 - DNA, 32 bp, Homo sapiens
  source 1..32: mol_type = other DNA
atatatatatatatatccaagaggatatatct

SEQ ID NO: 5 - DNA, 14 bp, Homo sapiens
  source 1..14: mol_type = other DNA
atatatatacacat

SEQ ID NO: 6 - DNA, 29 bp, Homo sapiens
  source 1..29: mol_type = other DNA
tatatatatcatccaagaggatatatata

SEQ ID NO: 7 - DNA, 28 bp, Homo sapiens
  source 1..28: mol_type = other DNA
ccaagaggatatatatatgtatatatat

SEQ ID NO: 8 - DNA, 30 bp, Homo sapiens
  source 1..30: mol_type = other DNA
ctctctccaagaggatatatatatatatat

SEQ ID NO: 9 - DNA, 34 bp, Homo sapiens
  source 1..34: mol_type = other DNA
ctctctacaagaggagatatatatatatatatat

SEQ ID NO: 10 - DNA, 25 bp, Homo sapiens
  source 1..25: mol_type = other DNA
ctccaagaggtatatatatatatat

SEQ ID NO: 11 - DNA, 31 bp, Homo sapiens
  source 1..31: mol_type = other DNA
ctatctccaagaggtatacatatatatatat

SEQ ID NO: 12 - DNA, 34 bp, Homo sapiens
  source 1..34: mol_type = other DNA
ctatctccaagaggtatatatatatataatatat

SEQ ID NO: 13 - DNA, 54 bp, synthetic construct
  source 1..54: mol_type = other DNA
cagcagcagcagcagcagcagcagcagcagcagcaacagccgccaccgccgccg

SEQ ID NO: 14 - DNA, 48 bp, synthetic construct
  source 1..48: mol_type = other DNA
cagcagcagcagcagcagcagcagcagcaacagccgccaccgccgccg

SEQ ID NO: 15 - DNA, 69 bp, Synthetic construct
  source 1..69: mol_type = other DNA
cagcagcagcagcagcagcagcagcagcagcagcagcagcagcagcagcaacagccgccaccgccgccg

SEQ ID NO: 16 - DNA, 21 bp, synthetic construct
  source 1..21: mol_type = other DNA
cagcagcaacagccgccgccg

SEQ ID NO: 17 - DNA, 24 bp, synthetic construct
  source 1..24: mol_type = other DNA
cagcagcagcagcaacagccgccg